(19) United States
(12) Patent Application Publication
(10) Pub. No.: US 2026/0241555 A1
(43) Pub. Date: Aug. 20, 2026

(54) SYSTEMS AND METHODS FOR CONTROLLING ELASTIC CABLE DRIVEN ROBOT JOINTS

(71) Applicant: Vicarious Surgical Inc., Waltham, MA (US)

(72) Inventors: Michael Eilenberg, Malden, MA (US); Ryan Fish, Playa Vista, CA (US); Pankaj Chopra, Medford, MA (US)

(21) Appl. No.: 19/469,643

(22) PCT Filed: Apr. 1, 2024
(86) PCT No.: PCT/US2024/022523

§ 371 Date: Sep. 26, 2025

Related U.S. Application Data
(60) Provisional application No. 63/456,359, filed on Mar. 31, 2023.

(51) Int. Cl. B25J 9/16; A61B 34/00; A61B 34/37; A61B 90/00; B25J 9/10; B25J 13/08

(52) U.S. Cl. CPC B25J 9/1641; A61B 34/37; A61B 34/71; B25J 9/1045; B25J 9/1633; B25J 13/085; A61B 2034/715; A61B 2090/066

(57) ABSTRACT

Systems and methods for controlling cable driven surgical robot joints are provided. For example, a system can include a robotic joint of a robotic arm, a first motor, a second motor, a cable coupled to the first motor and the second motor and operationally coupled to the robotic joint to control a position of the robotic joint, a memory storing one or more instructions, and a processor configured to or programmed to read the one or more instructions stored in the memory. The processor can be configured to determine an unacceptable amount of slack or tension in the cable. The processor can be further configured to execute the one or more instructions in the memory to adjust the first motor and the second motor to remove the unacceptable amount of slack in the cable.

### CROSS-REFERENCE TO RELATED APPLICATIONS

This application claims the benefit of a priority to U.S. Provisional application No. 63/456,359, filed Mar. 31, 2023, the contents of which are incorporated herein by reference in its entirety.

### BACKGROUND

Robotic systems (e.g., surgical robotic systems) permit a user (also described herein as an “operator” or a “user”) to perform an operation using robotically-controlled instruments to perform tasks and functions during a procedure. However, the commonly used kinematic (position-based) control of motors does not ensure appropriate tensions are maintained in the drive cables. This results in poor performance under various circumstances, such as external loading of a robotic arm of the robotic system, cable creep, and change in effective cable lengths due to cable unspooling or slip in transmission. The lack of appropriately maintained tension can cause cables to get tangled or caught, and may reduce performance.

### SUMMARY

A robotic surgical system is presented. The system can include a robotic joint of a robotic arm, a first motor, a second motor, a cable coupled to the first motor and the second motor and operationally coupled to the robotic joint to control a position of the robotic joint, a memory storing one or more instructions, and a processor configured to or programmed to read the one or more instructions stored in the memory. The processor can be configured or programmed to determine an unacceptable amount of slack or tension in the cable. The processor can be further configured or programmed to execute the one or more instructions in the memory to adjust the first motor and/or the second motor to remove the unacceptable amount of slack in the cable.

In some embodiments, the system can determine, in real time, a motor offset based at least in part on a model indicative of a relationship between a cable tension and a cable stretch associated with the cable over time. The system can adjust a motor angle associated with the first motor or the second motor based at least in part on the motor offset. The system can adaptively update the model in real time based at least in part on the motor offset. The system can update the model over time based at least in part on incoming cable tension and/or cable stretch.

In some embodiments, the system can receive a control scheme selection input. The system can further execute a control scheme, such as a position-limiting scheme, a parallel control scheme, or a hybrid control scheme. If the system operates in the position-limiting scheme, the system can determine a respective desired motor angle associated with the first motor and the second motor. The system can further adjust a respective motor angle associated with the first motor and the second motor to the respective desired motor angle. The motors associated with a joint operating under a position-limiting scheme can use a high-gain position controller with feedback from an encoder on each such motor. If the system operates in the parallel control scheme, the system can determine a respective desired motor torque associated with the first motor and the second motor based at least in part on a torque control loop and a feedforward control loop in parallel to the torque control loop. The feedforward control loop can provide additional motor torques to the first motor and the second motor to drive the first motor and the second motor toward a respective desired motor angle associated with the first motor and the second motor. The system can further adjust a respective motor torque associated with the first motor and the second motor to the respective desired motor torque. The motors associated with a joint operating under a parallel control scheme can use a high-gain torque controller with feedback from current sensing on each such motor. If the system operates in the hybrid control scheme, the system can automatically switch the motors associated with a joint operating under a hybrid control scheme between a position controller and a torque controller based at least in part on at least one of a respective desired motor angle associated with the first motor and the second motor, a velocity of the respective desired motor angle, or an agonist's applied force.

In some embodiments, the system can further execute an impedance control mode, which may be used as part of the control schemes including the position-limiting scheme, the parallel control scheme, and the hybrid control scheme. This impedance control mode can be used instead of, or in addition to one or more of the other two modes of motor operation (position mode or torque mode). The impedance mode can be used to produce motor behaviors in between the position and torque mode behaviors. For example, if it is desirable to limit a motor to not rotate past a given angle, position mode may be used to ensure this angle is not passed to the best of the motor's ability. If, instead, impedance control mode is used, the motor can allow some amount of motion past the angle limit according to the specified impedance or impedances. This allowance may be desirable in order to balance multiple conflicting commands, and/or to smooth out motor motions (e.g. reduce sudden jerks/stops/starts).

### BRIEF DESCRIPTION OF THE DRAWINGS

These and other features and advantages of the present invention will be more fully understood by reference to the following detailed description in conjunction with the attached drawings in which like reference numerals refer to like elements throughout the different views. The drawings illustrate principals of the invention and, although not to scale, show relative dimensions.

FIG. 1 is a diagram illustrating an example surgical robotic system in accordance with some embodiments.

FIG. 2A is an example perspective view of a patient cart including a robotic support system coupled to a robotic subsystem of the surgical robotic system in accordance with some embodiments.

FIG. 2B is an example perspective view of an example operator console of a surgical robotic system of the present disclosure in accordance with some embodiments.

FIG. 3A is a diagram illustrating an example side view of a surgical robotic system performing a surgery within an internal cavity of a subject in accordance with some embodiments.

FIG. 3B is a diagram illustrating an example top view of the surgical robotic system performing the surgery within the internal cavity of the subject of FIG. 3A in accordance with some embodiments.

FIG. 4A is an example perspective view of a single robotic arm subsystem in accordance with some embodiments.

FIG. 4B is an example perspective side view of a single robotic arm of the single robotic arm subsystem of FIG. 4A in accordance with some embodiments.

FIG. 5 is an example perspective front view of a camera assembly and a robotic arm assembly in accordance with some embodiments.

FIG. 6 is a flowchart illustrating a method for position-limiting carried out by a surgical robotic system in accordance with some embodiments.

FIG. 7A is a diagram illustrating an example of two independent motors driving a robotic joint using a position-limiting algorithm in accordance with some embodiments.

FIG. 7B is a diagram illustrating another example of two independent motors driving a robotic joint using a position-limiting algorithm in accordance with some embodiments.

FIG. 8 is a flowchart illustrating a method for adaptive position-limiting carried out by a surgical robotic system in accordance with some embodiments.

FIG. 9 is a flowchart illustrating a method for determining a motor offset carried out by a surgical robotic system in accordance with some embodiments.

FIG. 10 is a diagram illustrating an example model that can be used while a motor is an agonist to determine a motor offset in accordance with some embodiments.

FIG. 11 is a diagram illustrating an example model that can be used to estimate a slope in real time along with a motor offset in accordance with some embodiments.

FIG. 12A is a diagram illustrating a desired linear relationship to be extracted from data.

FIG. 12B is a diagram illustrating a bounding box for filtering out extremes in cable force and in cable stretch, where only data inside the box are used for estimation.

FIG. 12C is a diagram illustrating a result of filtering with several filters.

FIG. 12D is a diagram illustrating the same filtering as FIG. 12C but with a further constraint on a force-length slope.

FIG. 13 is a diagram illustrating an example hysteresis filter.

FIG. 14A is a diagram illustrating a parallel control scheme through a feedforward control loop in parallel to a motor torque control loop for each motor in accordance with some embodiments.

FIG. 14B is a diagram illustrating a parallel control scheme through a feedforward control loop in parallel to a motor torque control loop for each motor of multiple coupled joints in accordance with some embodiments.

FIG. 14C is a diagram illustrating an example of a mapping function in accordance with some embodiments.

FIG. 15 depicts a computing module in more detail in accordance with some embodiments.

### DETAILED DESCRIPTION

Controlling a robotic joint of a robotic arm of a robotic system with cables has its advantages, but cable-driven robotic joints and robotic arms also introduce problems associated with the fact that cables often have significant compliance. One example involves a robot joint being driven by a cable that forms a loop between a single motor and the robotic joint so that one-half of the cable (Cable A) drives the robotic joint at any given time while the opposite-half of the cable (Cable B) follows the robotic joint. If an obstruction impedes the motion of the robotic joint in the intended direction, Cable A stretches and Cable B develops slack. This slack generation can cause several problems. For example, a slackened cable can get caught on or in parts of the robotic system, pop off of idlers, and, depending on the maximum speed of the driving motors, the slack may take a significant amount of time to be taken up when trying to change the direction of the robotic joint. To facilitate explanation of the subject matter disclosed herein a surgical robot will be used as an example. However, the subject matter disclosed herein is equally applicable to other types of robots in fields other than surgical robots.

One of the benefits of this present disclosure is that it obviates the need for an explicit tensioning since control schemes as taught herein (e.g., position limiting scheme, parallel control scheme, and hybrid control scheme,) automatically adjust cable tensions and thus, no explicit tensioning is needed to remove unaccepted amount of slack in a cable.

Although the following descriptions detail mechanics and algorithms for joints and motors with rotational motion, it should be understood that the same challenges exist, and are solved by the methods herein, when the joints and/or motors actuate in linear motion with a compliant drivetrain. In particular, “angle” in a rotary context may be understood as “position” in a linear one. Although cable-driven robotic systems are emphasized herein, other drivetrains may utilize these same algorithms any time multiple motors act independently to reduce the effects of elastic and/or inelastic deformation has on the system performance (e.g. backlash in a traditional spur gear drivetrain due to clearances, or deflection through a plastic gear due to low stiffness relative to operational loads).

Additionally, in a well-designed robotic system, there exists a mapping function or matrix that maps joint angles to nominal motor angles such that, in the absence of forces, if the motors are driven to these nominal motor angles at all times, the cable lengths in the robotic system do not change as the robotic joint is driven, and thus, the tension or slack in the cables do not change. A conventional robotic system can be controlled in a straightforward manner by feeding a desired joint angle through the mapping function to achieve desired motor angles. However, when a robotic joint is put under load, this control strategy causes some cables to stretch and other cables to slacken. The stretching cables are desirable because they allow the robotic system to generate cable forces, and therefore robotic joint torques, but the slackening cables may be detrimental.

A surgical robotic system (also referred to as “system”) as taught herein can perform a method for independently controlling elastic cables attached to a robotic joint in a way that mitigates cable slack build-up without significantly affecting the joint performance. In some embodiments, the system as taught herein can include a position-limiting algorithm to prevent an antagonist motor from spooling out past a point at which its cable's tension would drop below a desired tension. An “agonist” as taught herein refers to a motor that is attempting to drive a joint toward a desired joint angle. An “antagonist” as taught herein refers to a motor that is attempting to maintain a desired amount of co-contraction force or slack in its cable. To apply the position-limiting algorithm, the mapping function can be used to compute nominal motor angles that would drive the robotic joint to a present joint position based at least in part on a motor offset as further described below. A total angle computed from the nominal motor angle and the motor offset can be a switching angle. The motors can then be commanded to move to desired motor angles (computed from a desired joint angle) or the switching angles (computed from present joint angle): whichever is spooled out less. If all motors are pre-tensioned to some desired cable tension at start-up and have an initial tension value, then the antagonist cable tensions can be maintained at this initial tension value instead of generating an uncontrolled amount of slack. For simplicity, the motor angles are set to read zero at the angles they reach at the end of this start-up. It should be understood that the motor angles can be set to any suitable values. Upon startup, motors can be commanded to pull on the robotic joint in their respective directions based on a predetermined co-contraction force. The positions of the motors can be then recorded relative to nominal motor angles, and motor angle readings can be shifted so that their measured angles match that of the nominal motor angles when in this co-contraction state.

Many control algorithms for cable-driven robots are cable-tension based. These algorithms directly control forces on the cables in order to avoid spooling out cables more than necessary. However, if cable tension cannot be measured or otherwise controlled, these force-control strategies might not be effective. A position-limiting algorithm as taught herein does not require explicit control of motor or cable forces. One advantage of controlling motor positions is that feedforward motor position commands may be readily incorporated to the controller, potentially speeding up the joint response.

Compared to existing position-based controllers, the position-limiting algorithm as taught herein can have several distinct features. The position-limiting algorithm as taught herein can have the ability to put an upper bound on slack generated by motors, even in the presence of external joint loads. In contrast, the conventional algorithms cannot prevent slack from building up in the cables under external loading of the joint(s). Additionally, the position-limiting algorithm as taught herein can have the ability to deliberately allow some slack to develop in antagonist's cables so as to improve joint tracking performance or enforce some tension in an antagonist's cable, because given friction in cables and robotic joints of a cable-driven robotic system, any forces on an antagonist's cable that can resist a motion of the robotic joint can be magnified by the capstan effect and cause the agonist motor(s) to work harder, possibly at a cost of joint linearity and/or tracking performance. A slack avoidance algorithm as taught herein can have the ability to adaptively adjust the allowed slack based on sensor data and/or models over time in real time or in near real time. In particular, slack avoidance may be any method that prevents a cable or cables in a cable-driven robot from developing unwanted slack, and optionally attempts to maintain a minimum cable tension on a set of cables

In some embodiments, the system as taught herein can include a robotic joint of a robotic arm, a first motor, a second motor, a cable coupled to the first motor and the second motor and operationally coupled to the robotic joint to control a position of the robotic joint, a memory storing one or more instructions, and a processor configured to or programmed to read the one or more instructions stored in the memory. The processor can be configured to determine an unacceptable amount of slack in the cable. The processor can be further configured to execute the one or more instructions in the memory to adjust the first motor and the second motor to remove the unacceptable amount of slack in the cable.

In controlling a cable-driven robot, it is important to manage the tensions in the cables so that cable tensions are maintained at desired levels and slack is not excessively generated. Generally, the motors for cable-driven robots may be controlled in several ways. Often control of cable-driven robot joints involves either the use of motor position mode control or motor torque mode control.

As taught herein, when driving a cable-driven robotic joint, at least one motor, (called an agonist), can accelerate the robotic joint in a desired direction. Meanwhile, at least one other motor (called an antagonist) can act opposite to an agonist to maintain tension in a following cable. An antagonist motor must follow the robotic joint along as the robotic joint moves so as not to provide too much resistance to an agonist motor. Further, an antagonist motor must not follow too quickly so as not to give slack to its cable. One way to maintain the appropriate behavior in an antagonist motor is to keep a small amount of tension on an antagonist cable. This can keep the antagonist cable from developing slack while also not adding too much resistance to the robotic joint or agonist motor.

When the robotic joint is commanded to move in a given direction, desired motor angles can be computed for all motors. Initially, any motor which is being commanded to spool in relative to its nominal motor angle can be considered to be an agonist and can be set into a position mode. Meanwhile, a motor that is being commanded to spool out relative to its nominal motor angle can be considered to be an antagonist, and it can continue to apply the desired co-contraction force (or other desired force) for tension-maintenance. However, some of the motor role switching below can allow motors to have roles that are briefly not consistent with the above definitions.

There are several ways to switch motors between agonist and antagonist roles. Some of the possible switching methods are described herein. A combination of these types of switches can also be implemented. When a switch happens, an agonist can become an antagonist, or vice versa. Therefore, a motor that was in position mode can swap to/from a torque mode. Note that the agonist and antagonist can transition at different times, especially if there are more than two motors per robotic joint. Therefore, for a two-motor robotic joint, there may be at any given time, two agonists or two antagonists if the switching does not happen simultaneously.

The systems as taught herein can utilize three different motor control modes, including a position mode, a torque mode, and an impedance control mode. The systems as taught herein can utilize the advantages of each of the position mode and the torque mode as they apply to a robotic joint.

A controller based only on position mode as taught herein can command all motors to enforce their own angular positions and can enforce tension in the cables and adapt to cable lengthening as needed via motor position commands. Conventional position mode-based controllers maintain co-contraction by assuming a fixed map of motor positions to joint positions, and thus, if the cables become longer over time through creep, plastic deformation, and the like, this mapping no longer holds, and the co-contraction tension falls over time. In the case where the motors are coupled to a robotic joint via non-rigid elements (stretchy cables, for example), a motor in the position mode as taught herein enables tuning of a joint controller without having to consider some factors of the motor itself, such as inertia, damping, friction, or the like. Further, the position mode as taught herein does not require re-tuning of a robotic joint controller for every motor coming off an assembly line. The position mode control can also allow for a feedforward control strategy where the motors are driven to the desired positions that correspond to the desired joint position, before adjusting for any joint position feedback. This feedforward control can also enable faster motion, since the controller does not have to wait for error to build up before applying a control effort.

The parallel control scheme as taught herein can command all motors to enforce their own torques and/or cable tensions and maintain cable tensions based on a feedforward control. This maintains co-contraction regardless of cable deformation, but has the disadvantage in that it is difficult to control the joint positions very quickly using a feedforward signal. A robotic joint may be position-controlled by feeding back the joint position error to produce individual motor torque commands so that the motor torques drive the joint in the desired direction. If, for example, the robotic joint needs to be driven in a positive direction, the motor(s) that drive the joint in that direction are commanded to produce more torque while the motor(s) that would drive the joint in the opposite direction are commanded to produce a smaller torque, such as a fixed tension-maintenance torque. In a conventional joint controller using torque mode motors, a motor cannot easily be commanded to snap quickly to a position computed from feedforward terms, since the motor control system must wait for the joint error signal to progress over time before the motor torque commands can be updated, as the bandwidth of a compliant drivetrain must limit the joint controller gain to preserve stability margins. Given this lack of feedforward control, the conventional joint controller based on torque mode motors may result in more sluggish responses in the joint as compared to a joint controller incorporating motor position feedback. The parallel control scheme as taught herein can provide a solution to the feedforward problem by adding a motor position controller in parallel to a motor torque controller for each motor. This parallel path can tune a controller that takes in present motor position, along with the feedforward joint command (mapped to motor space) and outputs a torque command to the motor to drive the motor to the position commanded by the feedforward controller. This torque is combined with the torque command from the closed-loop joint controller that is enforcing joint angle. By tuning controllers for both loops, the parallel control scheme as taught herein can apply feedforward position control of the joint to torque mode motors. In addition, using torque mode as taught herein can more directly control an amount of force a motor applies on a cable it drives and allows a user to specify how much cable tension will be applied during use.

In some embodiments, executing the parallel control scheme as taught herein may comprise executing a joint control algorithm based on a motor torque control loop with a parallel path of a feedforward control loop for position control or impedance control of motors to enable cable tension maintenance from the motor torque loop as well as feedforward response time from the feedforward control loop. In some embodiments, the executing the joint control algorithm may comprise: determining a respective desired motor torque associated with the first motor and the second motor based at least in part on a torque control loop and a feedforward control loop in parallel to the torque control loop, the feedforward control loop providing additional motor torques to the first motor and the second motor to drive the first motor and the second motor toward a respective desired motor angle associated with the first motor and the second motor; and adjusting a respective motor torque associated with the first motor and the second motor to the respective desired motor torque.

The hybrid control scheme as taught herein can take the advantages of both of the position mode scheme as taught herein and the torque mode scheme as taught herein, since at least one motor is in the position mode or is in the impedance control mode, driving a robotic joint quickly, while at least one other motor maintains the desired co-contraction tension in the torque mode. In some embodiments, the impedance control mode as taught herein can be used in place of the position mode scheme in a hybrid control scheme as taught herein. The hybrid control scheme as taught herein can combine the speed of motor-position based control with the ability to maintain co-contraction using motor torque. In some embodiments, the hybrid control scheme as taught herein can command some motors to enforce their own positions while others to enforce their own torques. The roles of the motors can change, i.e. a position mode motor may switch to being in torque mode and vice versa. In some embodiments, the hybrid control scheme as taught herein can control multiple motors that connect to the same robotic joint so that at least one motor is driven to control its own shaft angle (position mode), where another motor is driven to control its shaft torque or force that is applied to the robot joint (torque mode). The hybrid control scheme can make use of the advantages of each of these motor control modes. The position-based motor control can move the motor faster and without the explicit need for a joint level feedback, whereas the torque-based motor control allows for maintaining a desired tension in the cable to account for gradual cable stretch.

The impedance control mode can provide a motor control law that provides a relationship between any combination of positions and force/torque applied.

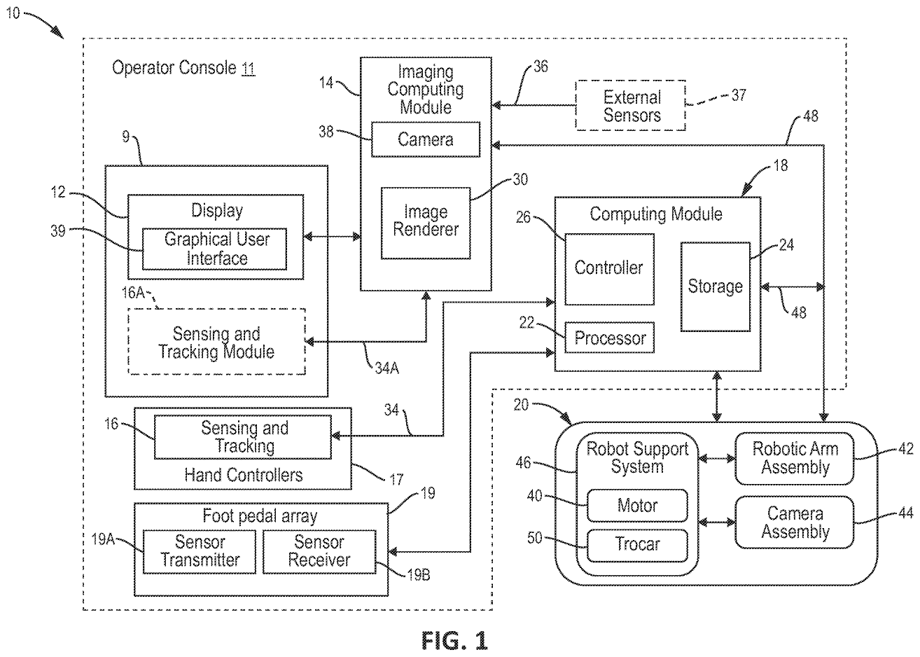

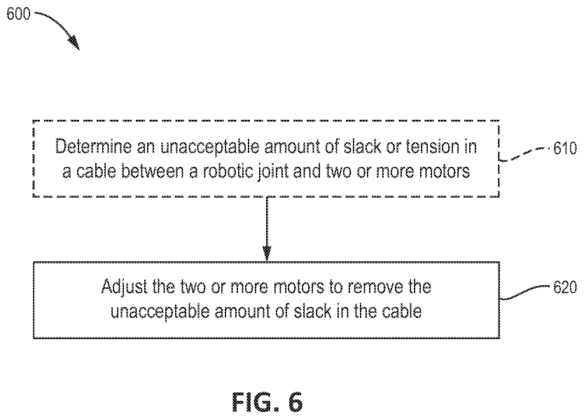

Prior to providing additional specific description of the position-limiting and control modes as taught herein with respect to FIGS. 6-15 a surgical robotic system in which some embodiments could be employed is described below with respect to FIGS. 1-5.

It should be understood that the rotary-motor cable drive as taught herein is for illustration. The present disclosure is not limited to a rotary-motor cable drive, but also applicable to linear-actuated cable drive, or any agonist/antagonist compliant drive. It should be also understood that the two-motor configuration as taught herein is for illustration. The present disclosure is not limited to the two-motor configuration, but also applicable to configurations having any number of motors (e.g., more than two motors). Further, it should be understood that the present disclosure can be applied to multiple cables that control multiple coupled robotic joints with non-unity transmission ratios.

While various embodiments have been taught and described herein, it will be clear to those skilled in the art that such embodiments are provided by way of example only. Numerous variations, changes, and substitutions can occur to those skilled in the art without departing from the invention. It can be understood that various alternatives to the embodiments taught herein can be employed.

As used herein, a “mapping function” refers to a mathematical relationship that relates a joint angles of a robotic joint with angles of one or more motors such that, if the cables were all infinitely stiff, the mapping function can predict nominal motor angles needed to achieve a given joint angle.

As used herein, a “nominal motor angle” refers to a motor angle that is obtained by feeding a present joint angle of a robotic joint through a mapping function into motor space.

As used herein, a “switching angle” refers to a motor angle beyond which a motor is not allowed to spool out.

As used herein, a “desired joint angle” refers to an angle to which a robotic joint is trying to be driven. The desired joint angle can be a present target angle of a robotic joint. This desired joint angle can include compensation to a target angle of a robotic joint for unmodeled disturbances.

As used herein, a “desired motor angle” refers to a motor angle that is obtained by feeding a desired joint angle through a mapping function into motor space.

A used herein, a “spooling out” refers to a motor rotation that drives cable off of a motor spool and into a robotic joint, which may reduce cable tension and potentially generating slack.

As used herein, a “spooling in” refers to a motor rotation that pulls cable from a robotic joint onto a motor's pulley, which may remove slack and potentially increasing cable tension.

As used herein, an “agonist” refers to a motor that is attempting to drive a joint toward a desired joint angle. Note that in a case of multiple motors controlling multiple joints, a given motor may be an “agonist” in regard to driving one joint but an “antagonist” for another joint.

As used herein, an “antagonist” refers to a motor that is attempting to maintain a desired amount of co-contraction force or slack in its cable. Note that in a case of multiple motors controlling multiple joints, a given motor may be an “agonist” in regard to driving one joint but an “antagonist” for another joint.

As used herein, a “model” refers to a mathematical representation of how quantities relate (e.g., a relationship between a cable stretch and a cable force, etc.).

As used herein, an “estimator” refers to an algorithm that uses measurements and a model to predict a quantity that is otherwise not directly measurable.

As used herein, a “torque mode” refers to a mode of motor control where a motor is commanded to apply a torque (e.g., approximated by electrical current).

As used herein, a “position mode” refers to a mode of motor control where a motor is commanded to go to a commanded position.

As used in the specification and claims, the terms “cable force” and “cable tension” can be used interchangeably. The terms “angle” and “position” can be used interchangeably.

As used in the specification and claims, the singular form “a,” “an,” and “the” include plural references unless the context clearly dictates otherwise. It will be further understood that the terms “comprises” and/or “comprising,” or “include” and/or “including,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Although some example embodiments can be described herein or in documents incorporated by reference as employing a plurality of units to perform example processes, it is understood that example processes can also be performed by one or a plurality of modules. Additionally, it is understood that the term controller can refer to a hardware device that includes a memory and a processor and is specifically programmed to execute the processes described herein in accordance with some embodiments. In some embodiments, the memory is configured to store the modules and the processor is specifically configured to execute said modules to perform one or more processes which are described further below. In some embodiments, multiple different controllers or controllers or multiple different types of controllers or controllers can be employed in performing one or more processes. In some embodiments, different controllers or controllers can be implemented in different portions of a surgical robotic systems.

### Surgical Robotic Systems

Some embodiments can be employed with a surgical robotic system. A system for robotic surgery can include a robotic subsystem. The robotic subsystem includes at least a portion, which can also be referred to herein as a robotic assembly herein, that can be inserted into a patient via a trocar through a single incision point or site. The portion inserted into the patient via a trocar is small enough to be deployed in vivo at the surgical site and is sufficiently maneuverable when inserted to be able to move within the body to perform various surgical procedures at multiple different points or sites. The portion inserted into the body that performs functional tasks can be referred to as a surgical robotic module, a surgical robotic module or a robotic assembly herein. The surgical robotic module can include multiple different submodules or parts that can be inserted into the trocar separately. The surgical robotic module, surgical robotic module or robotic assembly can include multiple separate robotic arms that are deployable within the patient along different or separate axes. These multiple separate robotic arms can be collectively referred to as a robotic arm assembly herein. Further, a surgical camera assembly can also be deployed along a separate axis. The surgical robotic module, surgical robotic module, or robotic assembly can also include the surgical camera assembly. Thus, the surgical robotic module, or robotic assembly employs multiple different components, such as a pair of robotic arms and a surgical or robotic camera assembly, each of which are deployable along different axes and are separately manipulatable, maneuverable, and movable. The robotic arms and the camera assembly that are disposable along separate and manipulatable axes is referred to herein as the Split Arm (SA) architecture. The SA architecture is designed to simplify and increase efficiency of the insertion of robotic surgical instruments through a single trocar at a single insertion site, while concomitantly assisting with deployment of the surgical instruments into a surgical ready state as well as the subsequent removal of the surgical instruments through the trocar. By way of example, a surgical instrument can be inserted through the trocar to access and perform an operation in vivo in the abdominal cavity of a patient. In some embodiments, various surgical instruments can be used or employed, including but not limited to robotic surgical instruments, as well as other surgical instruments known in the art.

The systems, devices, and methods taught herein can be incorporated into and/or used with a robotic surgical device and associated system taught for example in U.S. Pat. No. 10,285,765 and in PCT patent application Serial No. PCT/US2020/39203, and/or with the camera assembly and system taught in United States Publication No. 2019/0076199, and/or the systems and methods of exchanging surgical tools in an implantable surgical robotic system taught in PCT patent application Serial No. PCT/US2021/058820, where the content and teachings of all of the foregoing patents, patent applications and publications are incorporated herein by reference herein in their entirety. The surgical robotic module that forms part of the present invention can form part of a surgical robotic system that includes a user workstation that includes appropriate sensors and displays, and a robot support system (RSS) for interacting with and supporting the robotic subsystem of the present invention in some embodiments. The robotic subsystem includes a motor and a surgical robotic module that includes one or more robotic arms and one or more camera assemblies in some embodiments. The robotic arms and camera assembly can form part of a single support axis robotic system, can form part of the split arm (SA) architecture robotic system, or can have another arrangement. The robot support system can provide multiple degrees of freedom such that the robotic module can be maneuvered within the patient into a single position or multiple different positions. In one embodiment, the robot support system can be directly mounted to a surgical table or to the floor or ceiling within an operating room. In another embodiment, the mounting is achieved by various fastening means, including but not limited to, clamps, screws, or a combination thereof. In other embodiments, the structure can be free standing. The robot support system can mount a motor assembly that is coupled to the surgical robotic module, which includes the robotic arm assembly and the camera assembly. The motor assembly can include gears, motors, drivetrains, electronics, and the like, for powering the components of the surgical robotic module.

The robotic arm assembly and the camera assembly are capable of multiple degrees of freedom of movement. According to some embodiments, when the robotic arm assembly and the camera assembly are inserted into a patient through the trocar, they are capable of movement in at least the axial, yaw, pitch, and roll directions. The robotic arms of the robotic arm assembly are designed to incorporate and employ a multi-degree of freedom of movement robotic arm with an end effector mounted at a distal end thereof that corresponds to a wrist area or joint of the user. In other embodiments, the working end (e.g., the end effector end) of the robotic arm is designed to incorporate and use or employ other robotic surgical instruments, such as for example the surgical instruments set forth in U.S. Pub. No. 2018/0221102, the entire contents of which are herein incorporated by reference.

Like numerical identifiers are used throughout the figures to refer to the same elements.

FIG. 1 is a schematic illustration of an example surgical robotic system 10 in which aspects of the present disclosure can be employed in accordance with some embodiments of the present disclosure. The surgical robotic system 10 includes an operator console 11 and a robotic subsystem 20 in accordance with some embodiments.

The operator console 11 includes a display 12, an image computing module 14, which can be a three-dimensional (3D) computing module, hand controllers 17 having a sensing and tracking module 16, and a computing module 18. Additionally, the operator console 11 can include a foot pedal array 19 including a plurality of pedals. The display 12 can include a graphical user interface 39. The graphical user interface 39, the controller 26 or the image renderer 30, or both, can render one or more images or one or more graphical user interface elements on the graphical user interface 39. For example, a pillar box associated with a mode of operating the surgical robotic system 10, or any of the various components of the surgical robotic system 10, can be rendered on the graphical user interface 39. Also live video footage captured by a camera assembly 44 can also be rendered by the controller 26 or the image renderer 30 on the graphical user interface 39.

The operator console 11 can include a visualization system 9 that includes the display 12 which can be any selected type of display for displaying information, images or video generated by the image computing module 14, the computing module 18, and/or the robotic subsystem 20. The display 12 can include or form part of, for example, a head-mounted display (HMD), an augmented reality (AR) display (e.g., an AR display, or AR glasses in combination with a screen or display), a screen or a display, a two-dimensional (2D) screen or display, a three-dimensional (3D) screen or display, and the like. The display 12 can also include an optional sensing and tracking module 16A. In some embodiments, the display 12 can include an image display for outputting an image from a camera assembly 44 of the robotic subsystem 20.

The hand controllers 17 are configured to sense a movement of the operator's hands and/or arms to manipulate the surgical robotic system 10. The hand controllers 17 can include the sensing and tracking module 16, circuitry, and/or other hardware. The sensing and tracking module 16 can include one or more sensors or detectors that sense movements of the operator's hands. In some embodiments, the one or more sensors or detectors that sense movements of the operator's hands are disposed in the hand controllers 17 that are grasped by or engaged by hands of the operator. In some embodiments, the one or more sensors or detectors that sense movements of the operator's hands are coupled to the hands and/or arms of the operator. For example, the sensors of the sensing and tracking module 16 can be coupled to a region of the hand and/or the arm, such as the fingers, the wrist region, the elbow region, and/or the shoulder region. Additional sensors can also be coupled to a head and/or neck region of the operator in some embodiments. In some embodiments, the sensing and tracking module 16 can be external and coupled to the hand controllers 17 via electricity components and/or mounting hardware. In some embodiments, the optional sensor and tracking module 16A can sense and track movement of one or more of an operator's head, of at least a portion of an operator's head, an operator's eyes or an operator's neck based, at least in part, on imaging of the operator in addition to or instead of by a sensor or sensors attached to the operator's body.

In some embodiments, the sensing and tracking module 16 can employ sensors coupled to the torso of the operator or any other body part. In some embodiments, the sensing and tracking module 16 can employ in addition to the sensors an Inertial Momentum Unit (IMU) having for example an accelerometer, gyroscope, magnetometer, and a motion processor. The addition of a magnetometer allows for reduction in sensor drift about a vertical axis. In some embodiments, the sensing and tracking module 16 also include sensors placed in surgical material such as gloves, surgical scrubs, or a surgical gown. The sensors can be reusable or disposable. In some embodiments, sensors can be disposed external of the operator, such as at fixed locations in a room, such as an operating room. The external sensors 37 can generate external data 36 that can be processed by the computing module 18 and hence employed by the surgical robotic system 10.

The sensors generate position and/or orientation data indicative of the position and/or orientation of the operator's hands and/or arms. The sensing and tracking modules 16 and/or 16A can be utilized to control movement (e.g., changing a position and/or an orientation) of the camera assembly 44 and robotic arm assembly 42 of the robotic subsystem 20. The tracking and position data 34 generated by the sensing and tracking module 16 can be conveyed to the computing module 18 for processing by at least one processor 22.

The computing module 18 can determine or calculate, from the tracking and position data 34 and 34A, the position and/or orientation of the operator's hands or arms, and in some embodiments of the operator's head as well, and convey the tracking and position data 34 and 34A to the robotic subsystem 20. The tracking and position data 34, 34A can be processed by the processor 22 and can be stored for example in the storage 24. The tracking and position data 34 and 34A can also be used by the controller 26, which in response can generate control signals for controlling movement of the robotic arm assembly 42 and/or the camera assembly 44. For example, the controller 26 can change a position and/or an orientation of at least a portion of the camera assembly 44, of at least a portion of the robotic arm assembly 42, or both. In some embodiments, the controller 26 can also adjust the pan and tilt of the camera assembly 44 to follow the movement of the operator's head.

The robotic subsystem 20 can include a robot support system (RSS) 46 having a motor 40 and a trocar 50 or trocar mount, the robotic arm assembly 42, and the camera assembly 44. The robotic arm assembly 42 and the camera assembly 44 can form part of a single support axis robot system, such as that taught and described in U.S. Pat. No. 10,285,765, or can form part of a split arm (SA) architecture robot system, such as that taught and described in PCT Patent Application No. PCT/US2020/039203, both of which are incorporated herein by reference in their entirety.

The robotic subsystem 20 can employ multiple different robotic arms that are deployable along different or separate axes. In some embodiments, the camera assembly 44, which can employ multiple different camera elements, can also be deployed along a common separate axis. Thus, the surgical robotic system 10 can employ multiple different components, such as a pair of separate robotic arms and the camera assembly 44, which are deployable along different axes. In some embodiments, the robotic arm assembly 42 and the camera assembly 44 are separately manipulatable, maneuverable, and movable. The robotic subsystem 20, which includes the robotic arm assembly 42 and the camera assembly 44, is disposable along separate manipulatable axes, and is referred to herein as an SA architecture. The SA architecture is designed to simplify and increase efficiency of the insertion of robotic surgical instruments through a single trocar at a single insertion point or site, while concomitantly assisting with deployment of the surgical instruments into a surgical ready state, as well as the subsequent removal of the surgical instruments through the trocar 50 as further described below.

The RSS 46 can include the motor 40 and the trocar 50 or a trocar mount. The RSS 46 can further include a support member that supports the motor 40 coupled to a distal end thereof. The motor 40 in turn can be coupled to the camera assembly 44 and to each of the robotic arm assembly 42. The support member can be configured and controlled to move linearly, or in any other selected direction or orientation, one or more components of the robotic subsystem 20. In some embodiments, the RSS 46 can be free standing. In some embodiments, the RSS 46 can include the motor 40 that is coupled to the robotic subsystem 20 at one end and to an adjustable support member or element at an opposed end

The motor 40 can receive the control signals generated by the controller 26. The motor 40 can include gears, one or more motors, drivetrains, electronics, and the like, for powering and driving the robotic arm assembly 42 and the cameras assembly 44 separately or together. The motor 40 can also provide mechanical power, electrical power, mechanical communication, and electrical communication to the robotic arm assembly 42, the camera assembly 44, and/or other components of the RSS 46 and robotic subsystem 20. The motor 40 can be controlled by the computing module 18. The motor 40 can thus generate signals for controlling one or more motors that in turn can control and drive the robotic arm assembly 42, including for example the position and orientation of each robot joint of each robotic arm, as well as the camera assembly 44. The motor 40 can further provide for a translational or linear degree of freedom that is first utilized to insert and remove each component of the robotic subsystem 20 through the trocar 50. The motor 40 can also be employed to adjust the inserted depth of each robotic arm of the robotic arm assembly 42 when inserted into the patient 100 through the trocar 50.

The trocar 50 is a medical device that can be made up of an awl (which can be a metal or plastic sharpened or non-bladed tip), a cannula (essentially a hollow tube), and a seal in some embodiments. The trocar 50 can be used to place at least a portion of the robotic subsystem 20 in an interior cavity of a subject (e.g., a patient) and can withdraw gas and/or fluid from a body cavity. The robotic subsystem 20 can be inserted through the trocar 50 to access and perform an operation in vivo in a body cavity of a patient. In some embodiments, the robotic subsystem 20 can be supported, at least in part, by the trocar 50 or a trocar mount with multiple degrees of freedom such that the robotic arm assembly 42 and the camera assembly 44 can be maneuvered within the patient into a single position or multiple different positions. In some embodiments, the robotic arm assembly 42 and camera assembly 44 can be moved with respect to the trocar 50 or a trocar mount with multiple different degrees of freedom such that the robotic arm assembly 42 and the camera assembly 44 can be maneuvered within the patient into a single position or multiple different positions.

In some embodiments, the RSS 46 can further include an optional controller for processing input data from one or more of the system components (e.g., the display 12, the sensing and tracking module 16, the robotic arm assembly 42, the camera assembly 44, and the like), and for generating control signals in response thereto. The motor 40 can also include a storage element for storing data in some embodiments.

The robotic arm assembly 42 can be controlled to follow the scaled-down movement or motion of the operator's arms and/or hands as sensed by the associated sensors in some embodiments and in some modes of operation. The robotic arm assembly 42 include a first robotic arm including a first end effector at distal end of the first robotic arm, and a second robotic arm including a second end effector disposed at a distal end of the second robotic arm. In some embodiments, the robotic arm assembly 42 can have portions or regions that can be associated with movements associated with the shoulder, elbow, and wrist joints as well as the fingers of the operator. For example, the robotic elbow joint can follow the position and orientation of the human elbow, and the robotic wrist joint can follow the position and orientation of the human wrist. The robotic arm assembly 42 can also have associated therewith end regions that can terminate in end-effectors that follow the movement of one or more fingers of the operator in some embodiments, such as for example the index finger as the user pinches together the index finger and thumb. In some embodiments, while the robotic arm assembly 42 can follow movement of the arms of the operator in some modes of control while a virtual chest of the robotic assembly can remain stationary (e.g., in an instrument control mode). In some embodiments, the position and orientation of the torso of the operator are subtracted from the position and orientation of the operator's arms and/or hands. This subtraction allows the operator to move his or her torso without the robotic arms moving. Further disclosure control of movement of individual arms of a robotic assembly is provided in International Patent Application Publications WO 2022/094000 A1 and WO 2021/231402 A1, each of which is incorporated by reference herein in its entirety.

The camera assembly 44 is configured to provide the operator with image data 48, such as for example a live video feed of an operation or surgical site, as well as enable the operator to actuate and control the cameras forming part of the camera assembly 44. In some embodiments, the camera assembly 44 can include one or more cameras (e.g., a pair of cameras), the optical axes of which are axially spaced apart by a selected distance, known as the inter-camera distance, to provide a stereoscopic view or image of the surgical site. In some embodiments, the operator can control the movement of the cameras via movement of the hands via sensors coupled to the hands of the operator or via hand controllers 17 grasped or held by hands of the operator, thus enabling the operator to obtain a desired view of an operation site in an intuitive and natural manner. In some embodiments, the operator can additionally control the movement of the camera via movement of the operator's head. The camera assembly 44 is movable in multiple directions, including for example in yaw, pitch and roll directions relative to a direction of view. In some embodiments, the components of the stereoscopic cameras can be configured to provide a user experience that feels natural and comfortable. In some embodiments, the interaxial distance between the cameras can be modified to adjust the depth of the operation site perceived by the operator.

The image or video data 48 generated by the camera assembly 44 can be displayed on the display 12. In embodiments in which the display 12 includes an HMD, the display can include the built-in sensing and tracking module 16A that obtains raw orientation data for the yaw, pitch and roll directions of the HMD as well as positional data in Cartesian space (x, y, z) of the HMD. In some embodiments, positional and orientation data regarding an operator's head can be provided via a separate head-tracking module. In some embodiments, the sensing and tracking module 16A can be used to provide supplementary position and orientation tracking data of the display in lieu of or in addition to the built-in tracking system of the HMD. In some embodiments, no head tracking of the operator is used or employed. In some embodiments, images of the operator can be used by the sensing and tracking module 16A for tracking at least a portion of the operator's head.

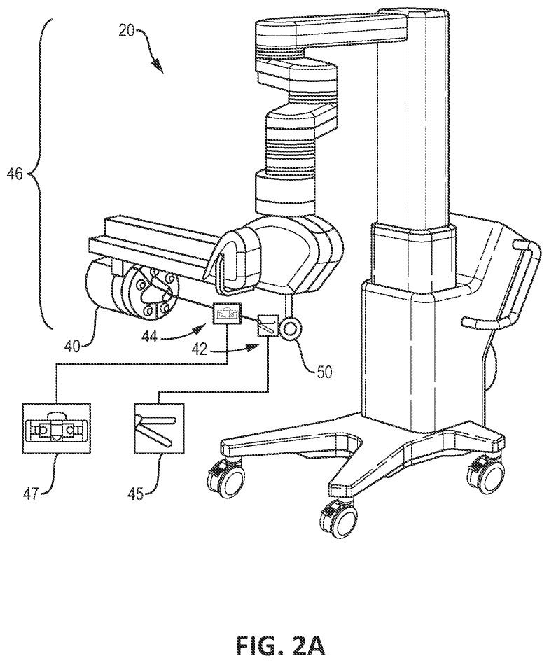

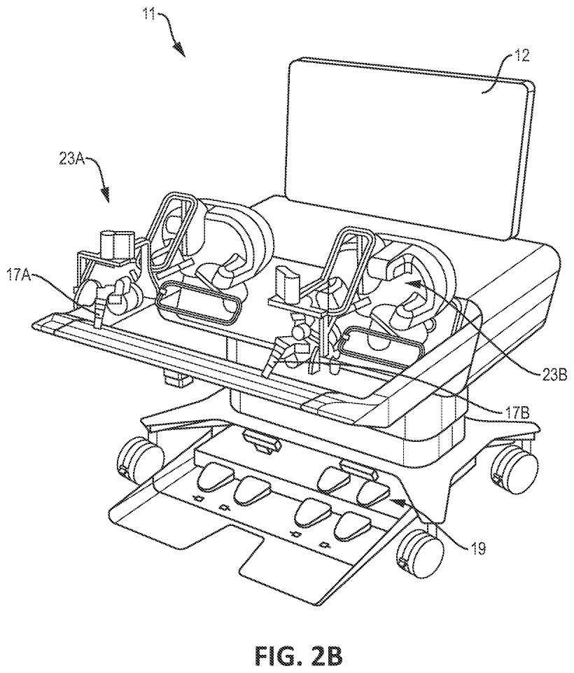

FIG. 2A depicts an example robotic assembly 20, which is also referred to herein as a robotic subsystem, of a surgical robotic system 10 incorporated into or mounted onto a mobile patient cart in accordance with some embodiments. In some embodiments, the robotic subsystem 20 includes the RSS 46, which, in turn includes the motor 40, the robotic arm assembly 42 having end-effectors 45, the camera assembly 44 having one or more cameras 47, and can also include the trocar 50 or a trocar mount.

FIG. 2B depicts an example of an operator console 11 of the surgical robotic system 10 of the present disclosure in accordance with some embodiments. The operator console 11 includes a display 12, hand controllers 17, and also includes one or more additional controllers, such as a foot pedal array 19 for control of the robotic arm assembly 42, for control of the camera assembly 44, and for control of other aspects of the system.

FIG. 2B depicts the left hand controller subsystem 23A and the right hand controller subsystem 23B of the operator console. The left hand controller subsystem 23A includes and supports the left hand controller 17A and the right hand controller subsystem 23B includes and supports the right hand controller 17B. In some embodiments, the left hand controller subsystem 23A can releasably connect to or engage the left hand controller 17A, and right hand controller subsystem 23B can releasably connect to or engage the right hand controller 17A. In some embodiments, the connections can be both physical and electronic so that the left hand controller subsystem 23A and the right hand controller subsystem 23B can receive signals from the left hand controller 17A and the right hand controller 17B, respectively, including signals that convey inputs received from a user selection on a button or touch input device of the left hand controller 17A or the right hand controller 17B.

Each of the left hand controller subsystem 23A and the right hand controller subsystem 23B can include components that enable a range of motion of the respective left hand controller 17A and right hand controller 17B, so that the left hand controller 17A and right hand controller 17B can be translated or displaced in three dimensions and can additionally move in the roll, pitch, and yaw directions. Additionally, each of the left hand controller subsystem 23A and the right hand controller subsystem 23B can register movement of the respective left hand controller 17A and right hand controller 17B in each of the forgoing directions and can send a signal providing such movement information to the processor 22 (as illustrated in FIG. 1) of the surgical robotic system 10.

In some embodiments, each of the left hand controller subsystem 23A and the right hand controller subsystem 23B can be configured to receive and connect to or engage different hand controllers (not illustrated). For example, hand controllers with different configurations of buttons and touch input devices can be provided. Additionally, hand controllers with a different shape can be provided. The hand controllers can be selected for compatibility with a particular surgical robotic system or a particular surgical robotic procedure or selected based upon preference of an operator with respect to the buttons and input devices or with respect to the shape of the hand controller in order to provide greater comfort and ease for the operator.

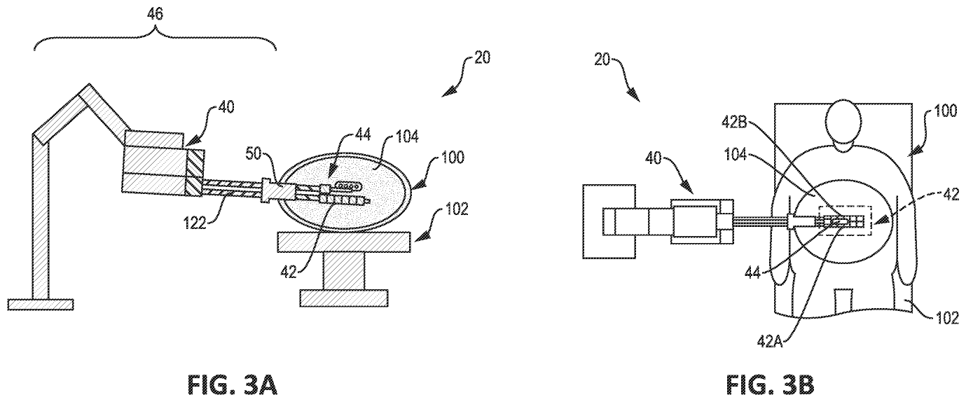

FIG. 3A schematically depicts a side view of the surgical robotic system 10 performing a surgery within an internal cavity 104 of a subject 100 in accordance with some embodiments and for some surgical procedures. FIG. 3B schematically depicts a top view of the surgical robotic system 10 performing the surgery within the internal cavity 104 of the subject 100. The subject 100 (e.g., a patient) is placed on an operation table 102 (e.g., a surgical table 102). In some embodiments, and for some surgical procedures, an incision is made in the patient 100 to gain access to the internal cavity 104. The trocar 50 is then inserted into the patient 100 at a selected location to provide access to the internal cavity 104 or operation site. The RSS 46 can then be maneuvered into position over the patient 100 and the trocar 50. In some embodiments, the RSS 46 includes a trocar mount that attaches to the trocar 50. The camera assembly 44 and the robotic arm assembly 42 can be coupled to the motor 40 and inserted individually and/or sequentially into the patient 100 through the trocar 50 and hence into the internal cavity 104 of the patient 100. Although the camera assembly 44 and the robotic arm assembly 42 can include some portions that remain external to the subject's body in use, references to insertion of the robotic arm assembly 42 and/or the camera assembly 44 into an internal cavity of a subject and disposing the robotic arm assembly 42 and/or the camera assembly 44 in the internal cavity of the subject are referring to the portions of the robotic arm assembly 42 and the camera assembly 44 that are intended to be in the internal cavity of the subject during use. The sequential insertion method has the advantage of supporting smaller trocars and thus smaller incisions can be made in the patient 100, thus reducing the trauma experienced by the patient 100. In some embodiments, the camera assembly 44 and the robotic arm assembly 42 can be inserted in any order or in a specific order. In some embodiments, the camera assembly 44 can be followed by a first robotic arm 42A of the robotic arm assembly 42 and then followed by a second robotic arm 42B of the robotic arm assembly 42 all of which can be inserted into the trocar 50 and hence into the internal cavity 104. Once inserted into the patient 100, the RSS 46 can move the robotic arm assembly 42 and the camera assembly 44 to an operation site manually or automatically controlled by the operator console 11.

Further disclosure regarding control of movement of individual arms of a robotic arm assembly is provided in International Patent Application Publications WO 2022/094000 A1 and WO 2021/231402 A1, each of which is incorporated by reference herein in its entirety.

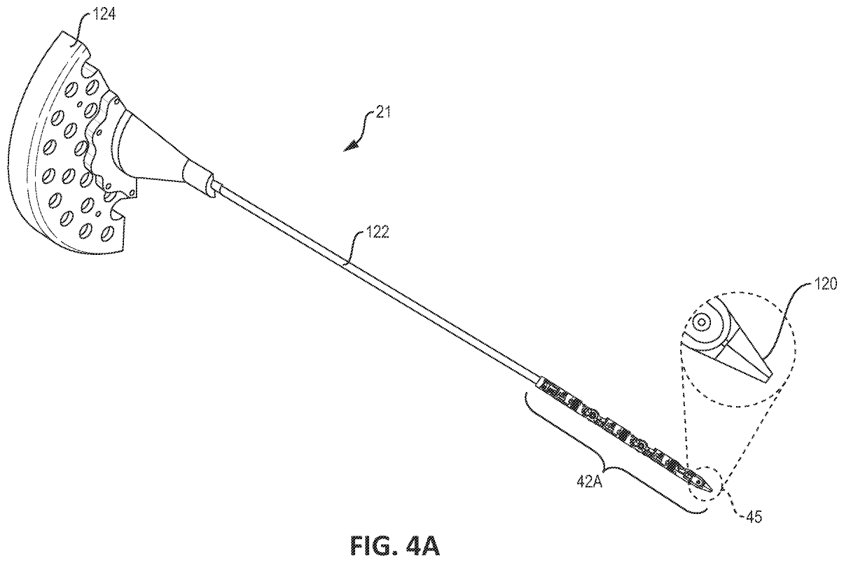

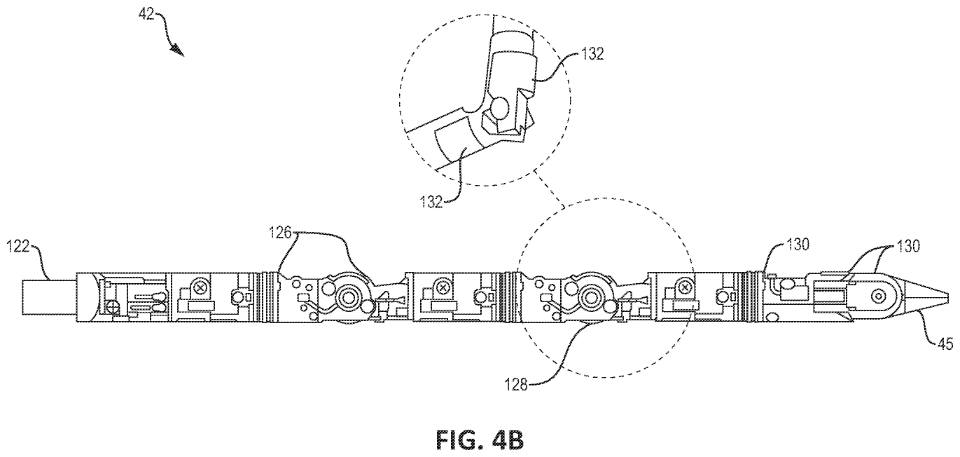

FIG. 4A is a perspective view of a robotic arm subassembly 21 in accordance with some embodiments. The robotic arm subassembly 21 includes a robotic arm 42A, the end-effector 45 having an instrument tip 120 (e.g., monopolar scissors, needle driver/holder, bipolar grasper, or any other appropriate tool), a shaft 122 supporting the robotic arm 42A. A distal end of the shaft 122 is coupled to the robotic arm 42A, and a proximal end of the shaft 122 is coupled to a housing 124 of the motor 40 (as illustrated in FIG. 2A). At least a portion of the shaft 122 can be external to the internal cavity 104 (as illustrated in FIGS. 3A and 3B). At least a portion of the shaft 122 can be inserted into the internal cavity 104 (as illustrated in FIGS. 3A and 3B).

FIG. 4B is a side view of the robotic arm assembly 42. The robotic arm assembly 42 includes a shoulder joint 126 forming a virtual shoulder, an elbow joint 128 having position sensors 132 (e.g., inductance based sensors or capacitive proximity sensors) and forming a virtual elbow, a wrist joint 130 forming a virtual wrist, and the end-effector 45 in accordance with some embodiments. The shoulder joint 126, the elbow joint 128, the wrist joint 130 can include a series of hinge and rotary joints to provide each arm with positionable, seven degrees of freedom, along with one additional grasping degree of freedom for the end-effector 45 in some embodiments. In some embodiments, the surgical robotic system 10 as a whole has nine degrees of freedom.

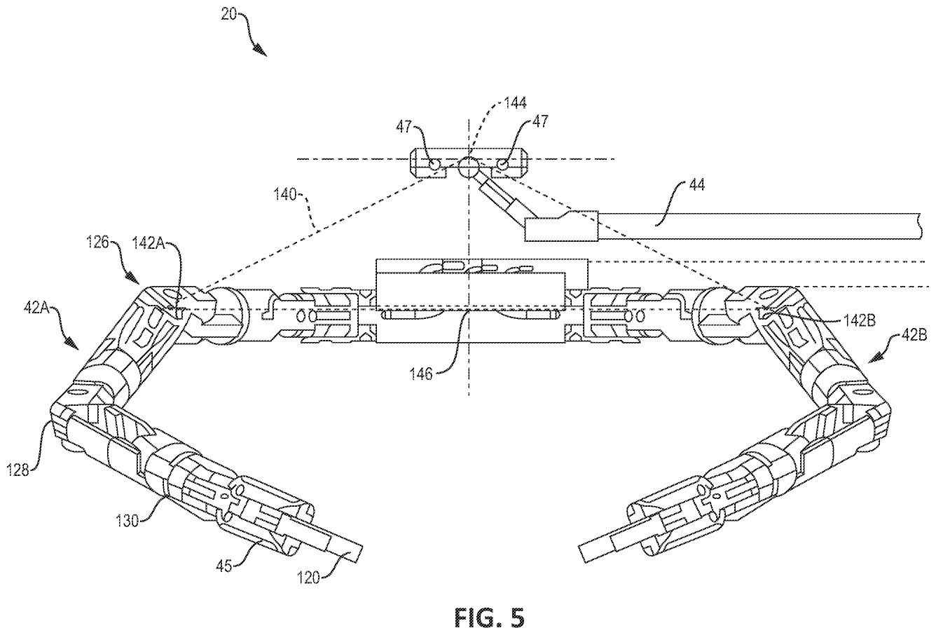

FIG. 5 illustrates a perspective front view of a portion of the robotic assembly 20 configured for insertion into an internal body cavity of a patient. The robotic assembly 20 includes a robotic arm 42A and a robotic arm 42B. The two robotic arms 42A and 42B can define, or at least partially define, a virtual chest 140 of the robotic assembly 20 in some embodiments. In some embodiments, the virtual chest 140 (depicted as a triangle with dotted lines) can be defined by a chest plane extending between a first pivot point 142A of a most proximal joint of the robotic arm 42A (e.g., a shoulder joint 126), a second pivot point 142B of a most proximal joint of the robotic arm 42B, and a camera imaging center point 144 of the camera(s) 47. A pivot center 146 of the virtual chest 140 lies in the middle of the virtual chest 140.

In some embodiments, sensors in one or both of the robotic arm 42A and the robotic arm 42B can be used by the surgical robotic system 10 to determine a change in location in three-dimensional space of at least a portion of each or both of the robotic arms 42A and 42B. In some embodiments, sensors in one or both of the first robotic arm 42A and second robotic arm 42B can be used by the surgical robotic system 10 to determine a location in three-dimensional space of at least a portion of one robotic arm relative to a location in three-dimensional space of at least a portion of the other robotic arm.

In some embodiments, the camera assembly 44 is configured to obtain images from which the surgical robotic system 10 can determine relative locations in three-dimensional space. For example, the camera assembly 44 can include multiple cameras, at least two of which are laterally displaced from each other relative to an imaging axis, and the system can be configured to determine a distance to features within the internal body cavity. Further disclosure regarding a surgical robotic system including camera assembly and associated system for determining a distance to features can be found in International Patent Application Publication No. WO 2021/159409, entitled “System and Method for Determining Depth Perception In Vivo in a Surgical Robotic System,” and published Aug. 12, 2021, which is incorporated by reference herein in its entirety. Information about the distance to features and information regarding optical properties of the cameras can be used by a system to determine relative locations in three-dimensional space.

### Position-Limiting Scheme

A position-limiting scheme as taught herein can be employed with any of the surgical robotic systems taught herein or any other suitable surgical robotic system. Further, some embodiments taught herein can be employed with semi-robotic endoscopic surgical systems that are only robotic in part.

The position-limiting scheme as taught herein can command all motors to enforce their own angular positions and can enforce tension in the cables and adapt to cable lengthening as needed via motor position commands. Position-limiting as taught herein can be used in the position mode to enforce tension in cables and adapt to cable lengthening as needed.

In the case where the motors are coupled to a robotic joint via non-rigid elements (stretchy cables, for example), the position-limiting scheme as taught herein can allow for a straightforward implementation of feedforward control, where the motor can be controlled to move very quickly to a nominal position where it would cause the robotic joint to be at a desired position. This feedforward control can be implemented by using a high-gain motor mode such as the position mode. Other modes are possible for this position-limiting scheme as well, such as impedance mode, although some of the benefits of the feedforward control would be lost, due to a likely slower response time of a lower-gain motor mode. In addition, a motor in the position mode, which operates and functions in accordance with the position-limiting scheme as taught herein can tune a joint controller without having to consider some factors of the motor itself, such as inertia, damping, friction, or the like. Further, the position mode as taught herein does not need to tune a robotic joint controller for every motor coming off an assembly line. The position mode can also allow for a feedforward control strategy where the motors are driven to the desired positions that correspond to the desired joint position, without the need for any joint position feedback. This feedforward control can also enable faster motion, since the controller does not have to wait for error to build up before applying a control effort.

In some embodiments, executing the position-limiting scheme may include determining, in real time, a motor offset based at least in part on a model indicative of a relationship between a cable tension and a cable stretch associated with the cable over time. In some embodiments, the system can update the model over time based at least in part on incoming cable tension and/or cable stretch.

In some embodiments, executing the position-limiting scheme may include determining a desired joint angle associated with the first motor and the second motor, and adjusting the angle of the first motor and the second motor to drive the robotic joint to reach the respective desired joint angle

If multiple motors control multiple joints in a coupled manner (more than two motors per joint and/or more than two joints per motor), the position mode control of motors may be used to control the joints by using superposition: for a given motor, the desired motor angle is calculated by summing the partial desired motor angles across all joints controlled by that motor, with each partial desired motor angle from a given joint calculated with the assumption that all other joints are already at their desired angles

The position-limiting scheme as taught herein can provide a slack-avoidance method that allows for a controlled amount of slack or tension and also provide an adaptive slack-avoidance method that adjusts the slack-avoidance output to account for changes in the cable force-length relationship in real time or in near real time, as further described below.

The position-limiting scheme as taught herein can be employed with any of the surgical robotic systems taught above or any other suitable surgical robotic system. Further, some embodiments taught herein can be employed with semi-robotic endoscopic surgical systems that are only robotic in part.

The position-limiting scheme as taught herein can be understood with reference to embodiments depicted in FIGS. 6-13 described below. For convenience, like reference numbers are used to reference similar features of the various embodiments illustrated in the figures, unless otherwise noted.

FIG. 6 is a flowchart illustrating a method 600 for a position-limiting scheme carried out by the surgical robotic system 10 in accordance with some embodiments. FIG. 6 is a flowchart based on a robotic joint coupled to two motors. Nonetheless, the process associated with the flowchart of FIG. 6 is applicable to a robotic joint coupled to more than two motors.

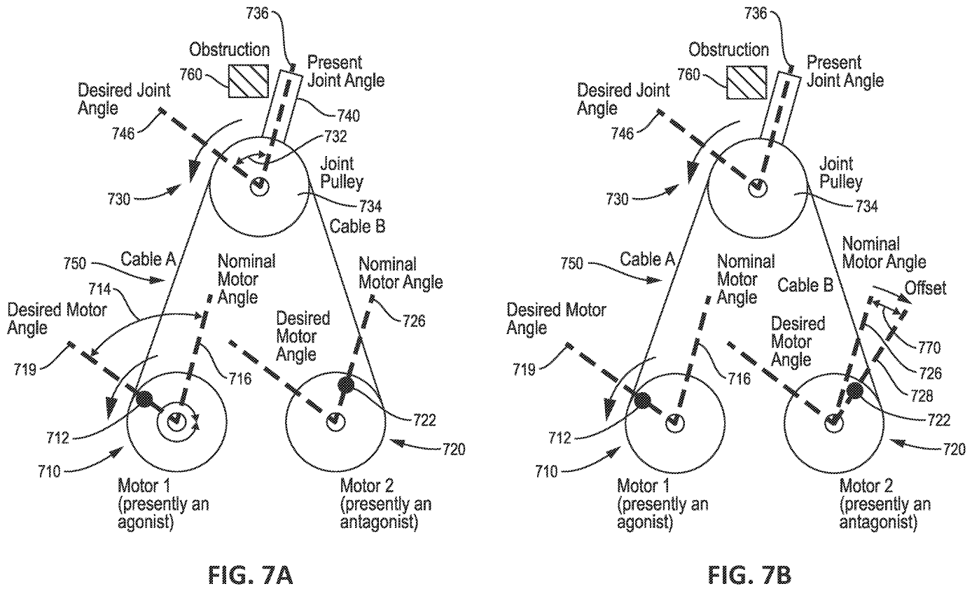

In step 610, the surgical robotic system 10 can determine an unacceptable amount of slack or tension in a cable between a robotic joint and two or more motors. For example, the cable can be coupled to a first motor and a second motor and operationally coupled to the robotic joint to control a position of the robotic joint. Examples are described with respect to FIGS. 7-12. In some embodiments, for example, in the parallel control schemes and the hybrid schemes, motors in the torque mode may automatically remove slack by applying torques and thus applying tension to the cables, without determining an unacceptable amount of slack.

In step 620, the surgical robotic system 10 can adjust the two or more motors to remove the unacceptable amount of slack in the cable using a position-limiting algorithm or an adaptive position-limiting algorithm as taught herein. Examples are described with respect to FIGS. 7-12.

In some embodiments, adjusting the one or more motors to remove the unacceptable amount of slack in the cable may include executing the robotic joint control algorithm that includes the slack avoidance algorithm that allows for a controlled amount of slack (positive or negative) for motors in position control.

In some embodiments, adjusting the one or more motors to remove the unacceptable amount of slack in the cable may include performing an adaptive slack avoidance that adjusts the slack avoidance algorithm's parameters based at least in part on sensor data.

FIG. 7A is a diagram illustrating an example of two independent motors 710 and 720 driving a robotic joint 730 using a position-limiting algorithm in accordance with some embodiments. FIG. 7B is a diagram illustrating another example of two independent motors 710 and 720 driving a robotic joint 730 using a position-limiting algorithm in accordance with some embodiments.

With regards to position-limiting, a motor is considered an agonist (with regard to a given joint) if the desired motor angle is spooled in past a switching angle.

With regards to position-limiting, a motor is considered an antagonist (with regard to a given joint) if the desired motor angle is spooled out past a switching angle.

FIGS. 7A and 7B illustrate that an example of a position-limiting scheme as taught herein that involves two independent motors 710 and 720 driving a robotic joint 730 of the same pulley diameter so that a mapping function is unity (each motor's angle maps to a joint angle 1:1). The motors 710 and 720 can be initially tensioned against each other so that portions (e.g., Cable A and Cable B) of a cable 750 have an equal amount of tension force, as desired. As illustrated, the robotic joint 730 can be commanded to rotate counterclockwise so that the arm or portion of an arm as represented by bar 740 moves to the left. The actual driven motor angles 712 and 722 are indicated by dots on respective pulleys. As illustrated, the motor 710 can be an agonist because it is being commanded to move the robotic joint 730 to the left, requiring it to spool in past a joint angle 732. Conversely, the motor 720 can be an antagonist because it is being commanded to spool out past a joint angle 732.

Without the position-limiting scheme as taught herein, the following occurs: when no forces are exerted on the robotic joint 730, for example, absent an obstruction in a movement path (rotational path or linear path), and in absence of other influences like friction, the cable 750 does not stretch, and both motors 710 and 720 may move together as if they were on the same rigid shaft. Both motors 710 and 720 would move together to the same counterclockwise angle as the robotic joint command, as illustrated by the motor angle 712 on the motor 710, regardless of a joint motion. If the robotic joint 730 became stuck on an obstruction 760 and the motors 710 and 720 continue to drive, Cable A stretches, allowing the motor 710 to drive the robotic joint 730 against the obstruction 760, but Cable B would reduce its tension force and eventually develop slack.

With the position-limiting scheme as taught herein, instead of both motors 710 and 720 always moving together, the motors 710 and 720 can be controlled (in absence of a motor offset as described below) so that no motor pulley is ever spooled out further than its switching angle (but the motor 710 or 720 may spool in as far as it is commanded). As taught herein, an antagonist cable (e.g., Cable B in this case) can remain at the same tension as it was at an initial tensioning step. In an example where the obstruction 760 is in place, the motor 710 may drive as far counterclockwise as a desired motor angle 714, but the position-limiting scheme as taught herein can prevent a motor 720 from rotating counterclockwise as long as the robotic joint 730 is stuck against the obstruction 760. If the obstruction 760 gives way and the robotic joint 730 begins to move past the obstruction 760, the motor 720 spools out at the same rate as the robotic joint 730.

In addition, the motor angles 712 and 722 indicated by the dots initially lined up with the bar 740 on the joint pulley 734 when the cable 750 was first tensioned. The nominal motor angle 716 or 726 can be the same as the present joint angle 736 because the pulley radii are all the same and the two motors 710 and 720 drive the robotic joint 730 (1:1 joint-motor mapping). The motor 720 (presently antagonist motor) can be limited by position-limiting to not spool out beyond its nominal motor angle 726 (illustrated in FIG. 7A) or a switching angle 728 (illustrated in FIG. 7B). Meanwhile, the motor 710 (presently agonist motor) spools in to the desired motor angle 719 to drive the robotic joint 730 to a desired joint angle 746 past the obstruction 760.

In some embodiments, the motors 710 and 720 can be commanded to an offset 770 to adjust their nominal motor angles, thus allowing the motors 710 and 720 to spool out to switching angles beyond their nominal motor angles to provide a controlled amount of slack, or spool in more than the nominal motor angles to provide extra tension for maintaining extra co-contraction force in the cable 750. This offset 770 can be set as a constant, or be a function of other variables (time, motor speed, joint acceleration, etc.), as desired.

In some embodiments, the position-limiting scheme as taught herein can be applied to multiple cables that control multiple coupled robotic joints with non-unity transmission ratios. Compared with a single robotic joint as illustrated FIGS. 7A and 7B, the difference lies in that the computation of nominal motor angles involves a matrix so that the mapping function can use multiple nominal joint angles in computing a nominal motor angle and/or have multiple nominal motor angles affected by a nominal joint angle. Similarly, the desired motor angles can be computed using multiple desired joint angles as input.

In some embodiments, the position-limiting scheme as taught herein can provide motor position commands that aim to achieve a desired amount of cable tension (or slack) when a motor is an antagonist. The position-limiting scheme method can be based on a fixed mapping from a motor angle to a joint angle, such that if the motor angle is driven consistently according to an initial joint-motor mapping function, the slack (or tension) of the antagonist's cable remains constant.

### On-Line Adaptation

In some embodiments, it is possible for a cable's rest-length to change over time or as a function of other factors. Cable materials can exhibit creep, and stress relaxation; terminations can slip. The adaptive position-limiting scheme as taught herein can provide a way to adjust a motor offset to account for the above changes over time in real time or in near real time.

In some embodiments, by implementing the adaptive avoidance method as taught herein, the system can collect and process sensor data, and then use the processed data to develop a model. The system can further generate an estimator to use the model to predict a motor offset (relative to a nominal motor angle) to provide a switching angle that achieves a desired cable force, when a motor is driven to that switching angle. This predicted offset can be then used to update an offset used in the system.

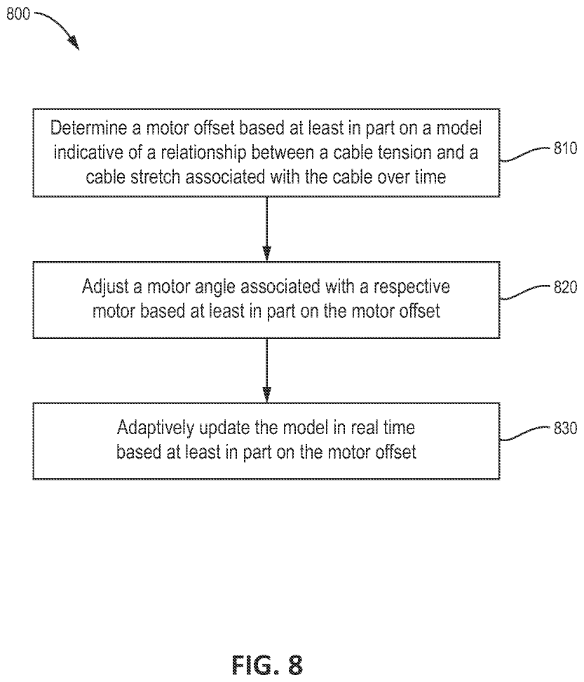

FIG. 8 is a flowchart illustrating a method 800 for adaptive position-limiting schemes carried out by the surgical robotic system 10 in accordance with some embodiments.

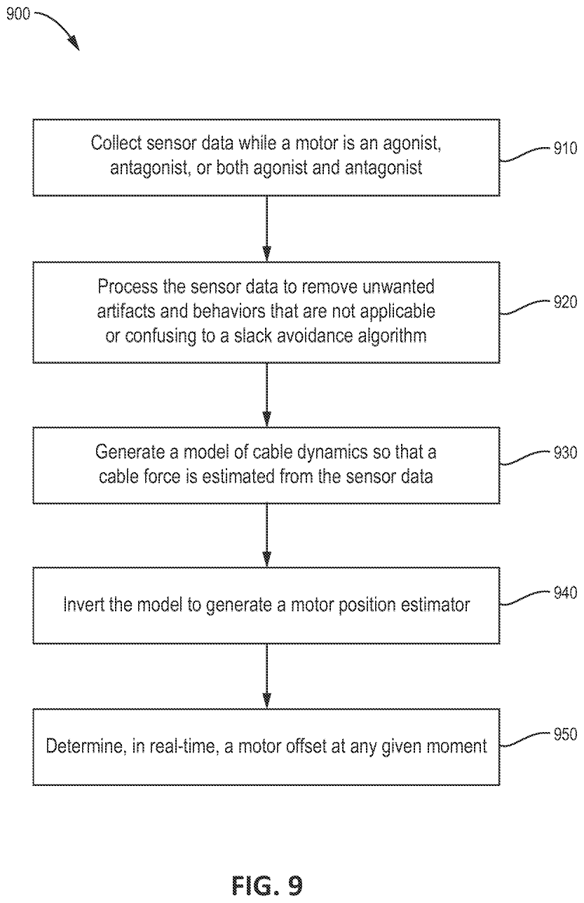

In step 810, the surgical robotic system 10 can determine a motor offset based at least in part on a model indicative of a relationship between a cable tension and a cable stretch associated with the cable over time. Examples are described with respect to FIGS. 9-12.

In step 820, the surgical robotic system 10 can adjust a motor angle associated with the first motor or the second motor based at least in part on the motor offset. For example, as illustrated in FIG. 7B, the surgical robotic system 10 can adjust a motor angle associated with the motor 720 by rotating the nominal motor angle 726 by the motor offset (that can be an angle) to reach to the switching angle 728.

If multiple motors control multiple joints in a coupled manner (more than 2 motors per joint and/or more than 2 joints per motor), the definition of agonist and antagonist are on a per-joint basis. Therefore, a motor may be an agonist for one joint and an antagonist for another joint. Likewise, a joint might have more than one agonist and/or more than one antagonist. The position-limiting scheme may therefore limit motor angles based on switching angles as they apply to one joint, but not limit motor angles as they apply to another joint. In other words, for a given motor, the position-limiting scheme operates on each joint independently; a motor's switching angle is computed for each joint that the motor controls, and the motor angle is limited based on that switching angle only in regards to the given joint. As described above, the total motor angle to which the motor is commanded is then the sum of the motor angles across all joints the motor controls.

In step 830, the surgical robotic system 10 can adaptively update the model in real time based at least in part on the motor offset. Examples are described with respect to FIGS. 9-12.

FIG. 9 is a flowchart illustrating a method 900 for determining a motor offset carried out by the surgical robotic system 10 in accordance with some embodiments.

In step 910, the surgical robotic system 10 can collect sensor data while a motor is an agonist, antagonist, or both agonist and antagonist. For example, the surgical robotic system 10 can use sensors (e.g., mounted on the motors, robotic joints and/or any suitable positions associated with robotic arms) to estimate a cable force and cable stretch states of a cable. In some embodiments, the sensors can detect a motor's electrical current, torque, and/or other force to estimate tension that the motor applies to the cable, while an encoder on the motor and another on the robotic joint can be used to estimate cable stretch.

In some embodiments, when the motor is an agonist motor, a cable-force-length model (and hence the estimator derived from that model) can be updated by monitoring the developed cable force on the cable as the agonist pulls the robotic joint against loads. Many different models can be used for the force-length relationship. For example, the surgical robotic system 10 can generate a model by a linear fit, which can be applied to systems with hysteresis and other artifacts by pre-filtering the incoming stretch and tension data as described below. Conversely, when the motor is an antagonist, the cable force is unlikely to achieve high values since, by design, the antagonist motor achieves the desired tension generated position-limiting. Still, the position-limiting algorithm can monitor the antagonist's cable force and compare that cable force to a target cable force for the antagonist. If the cable force is higher or lower than the target cable force, the motor offset can be adjusted accordingly.

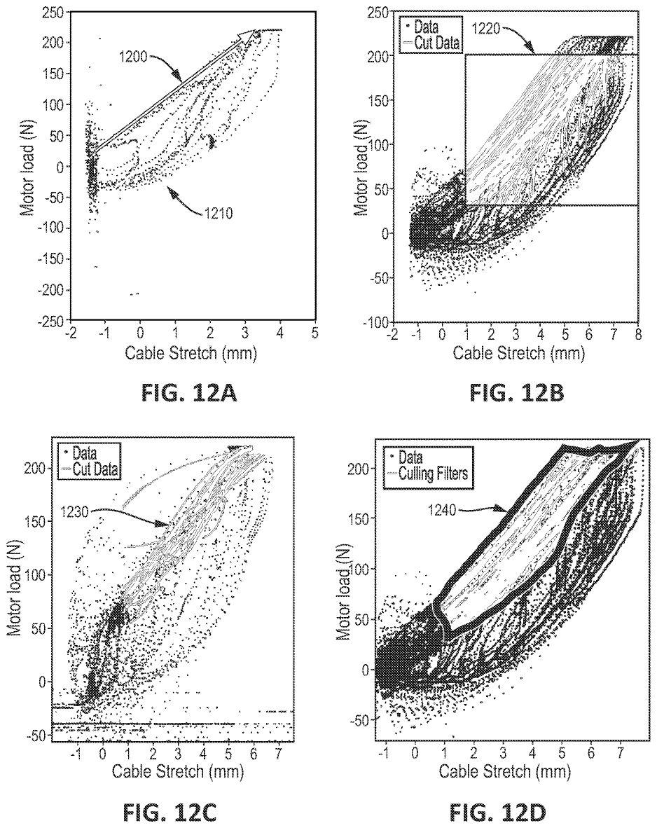

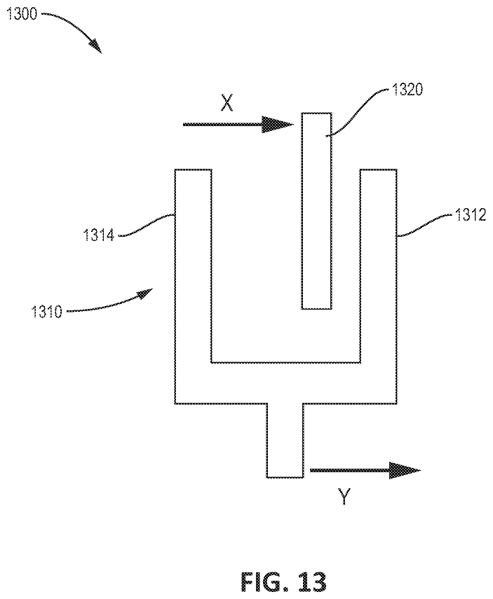

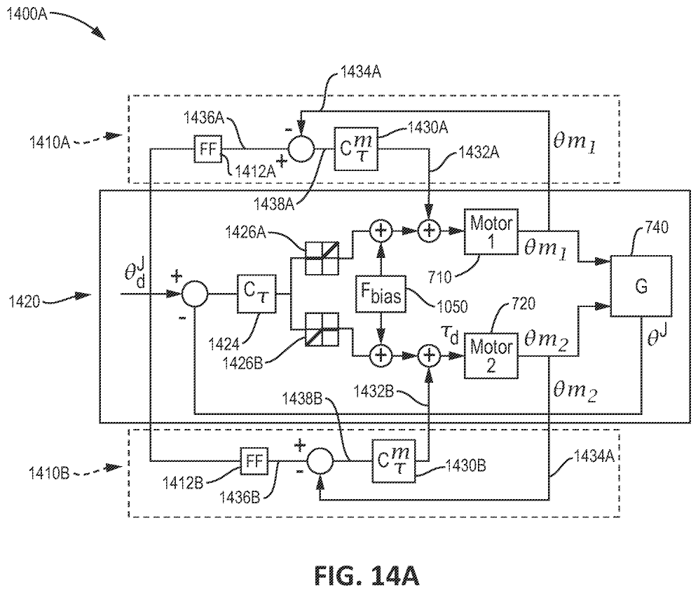

In step 920, the surgical robotic system 10 can process the sensor data to remove unwanted artifacts and behaviors that are not applicable or confusing to the position-limiting algorithm. For example, a force-length relationship of a cable may not fit well to a simple functional model because of hysteresis and other dynamics. The force to cable stretch relationship may not be 1:1 (as illustrated FIG. 12A). In such cases, processing sensor data can be useful to allow the model and estimator to be relatively simple. In the case of hysteresis, hysteresis models can be used to either model the force-length relationship directly, or as part of the filtering as in FIG. 12. Examples are described with respect to FIGS. 12 and 13.

In step 930, the surgical robotic system 10 can generate a model of cable dynamics, for example, cable tension and/or cable stretch so that a cable force can be estimated from the sensor data. The model can depend on whether the motor is an agonist or antagonist. The model can be set up to relate cable stretch to cable force, given a joint angle and any other relevant sensor data.

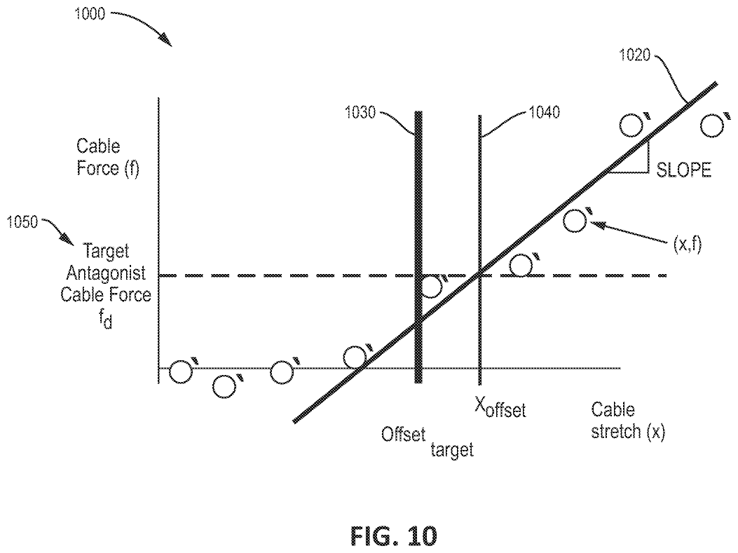

In some embodiments, using the sensor data, the surgical robotic system 10 can estimate the cable's force-length relationship. From this relationship, the surgical robotic system 10 can maintain an estimate of the amount of cable stretch at which the cable force is a desired value. If, for example, it can be desired that the antagonist motor's cable can be at about 40 Newtons of cable tension, the surgical robotic system 10 can compute the cable stretch at which the cable is expected to hit that tension. From this target cable stretch and measured joint angle, the desired motor angle that would produce the desired 40 Newtons of cable tension can then be determined from the joint-motor mapping function. An example of this procedure using a linear force-length model is illustrated in FIG. 10 as described below.

In step 940, the surgical robotic system 10 can invert the model to generate a motor position estimator so that given a desired cable force, the cable stretch, and hence, motor offset can be computed. The estimator can run in real-time to determine the motor offset at any given moment. Note that even if some amount of slack may be needed for an antagonist, the model can be used to predict the motor offset at which slack begins to develop, and thus, what the motor offset should be to generate the desired amount of slack. Examples are described with respect to FIGS. 10-12.

In step 950, the surgical robotic system 10 can determine, in real-time, a motor offset at any given moment.

FIG. 10 is a diagram illustrating an example estimator 1000 that can be used while a motor is an agonist to determine a motor offset in accordance with some embodiments. A Target Antagonist Cable Force f_{d} 1050 is an input to the estimator and is the desired to-contraction force for the given motor. As illustrated in FIG. 10, the model 1020 illustrates a relationship between a subset of the cable force (f) and a cable stretch (x) data by a linear fit. Note that other types of models may be used instead of linear model (e.g., quadratic, exponential, etc.) to represent the force-length behavior of the cable. A data point can be represented as (x, f). An instantaneous offset X_{offset} 1040 and an Offset_{target} 1030 can be determined by Equations listed below:

$${X}_{offset} = x - \frac{f - {f}_{d}}{SLOPE} \qquad ( 1 )$$

$${Offset}_{target} = moving\_average ( {X}_{offset} ) \qquad ( 2 )$$

Given the model parameters at a given time, the target antagonist cable force f_{d} can be used along with the model to determine an instantaneous offset X_{offset}. The instantaneous offset X_{offset} represents the motor offset that can achieve a target cable co-contraction force while the motor is an antagonist, as estimated at the given time. The Offset_{target} represents a moving average of the X_{offset}. The Offset_{target} can be used as the motor offset in order to smooth out adjustments of the motor offset over time. Note that the data points (x, f) can be instantaneous estimates of cable displacement and joint force, respectively.

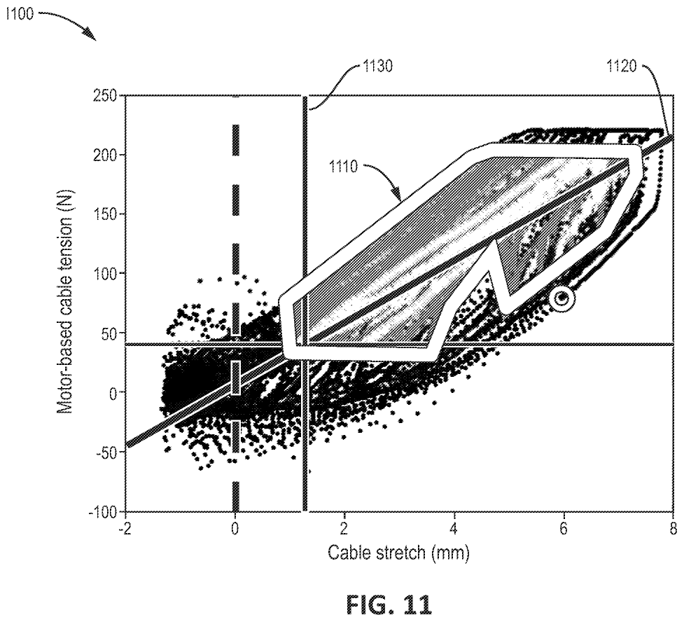

FIG. 11 is a diagram illustrating the estimator 1000 that can be used to estimate a slope in real time along with a motor offset in accordance with some embodiments. FIG. 11 illustrates the adaptive position-limiting scheme adapting to changes in cable rest-length and cable stiffness in real-time. The multiple plotted dots can represent raw (cable stretch, cable tension) data. Portion 1110 can represent the more recent data used by the moving-average as described above, a line 1120 can represent an instantaneous linear model, for example, as in the model 1020 of the cable force-length curve, and a vertical solid line 1130 can represent a motor offset (either the X_{offset} or the Offset_{target}) derived from the model 1120.

In some embodiments, the slope of the linear fit model, for example, the model 1000 can be assumed to be a constant, known value, or can be estimated in real-time along with the motor offset X_{offset} (e.g., illustrated in FIG. 12). In some embodiments, if the slope is assumed to be a constant value, leaving the motor offset to be estimated.

The estimator 1000 may provide a methodology for reducing computational complexity of the model while maintaining or even increasing its length-prediction power. In some embodiments, if the slope is estimated in real-time along with the motor offset, it may be advantageous to take into account the continuity of the force-length data when the cable is lengthening over time. For example, a slope can be more accurately estimated by grouping incoming data into blocks where, in each block, the slope and motor offset parameters can be reasonably consistent. A block may be a collection of force-length data points representing: a) one or more cycles of increasing and decreasing tension, b) solely half of a cycle, c) just the increasing-tension portion, or d) just the decreasing-tension portion. Slope and motor offset can then be estimated for each of those blocks. The slope and offset for the model can then be generated by an average of those individual slopes and motor offsets, weighted, for example, by the number of data points in each of the blocks, and/or, in a time-varying estimate by a time-weighted average. This can allow for compression of the dataset for use on a resource-constrained system, or for real-time update of the model to improve computational efficiency and reduce memory usage, instead of storing and fitting against all data points that is too computationally intensive or memory intense.

In some embodiments, the collection of force-length data points may be converted to slope and motor offset when the motor changes direction. In some embodiments, a hysteresis filter is used to determine this change. For example, the data are split into groups of data points, where the data points within each group are from a single stretching event of a cable. Then, memory containing previously collected data points can be freed, allowing a process to run on, for example, a resource-constrained processor(s) in an embedded systems for motor control. The data points of slope and motor offset may be stored with additional metadata including weighting information, such as timestamps (in order to weight recent data more heavily and delete old data (partially or entirely) or a number of points that made up fit as an indicator of data quality.

The data (compressed in some embodiments) can be stored as a series of models, each with their own model parameters, such as slope and intercept for a linear fit, and any metadata, can then be used to determine one or more X_{offset} values for each model. This calculation of each X_{offset} value may be performed by finding the intersection of each model with the Target Antagonist Cable Force f_{d}, or alternatively, creating a single model based on some averaging or statistical method over each of the stored model parameter sets. An X_{offset} thus found can then be used, possibly combined with other X_{offset} values and according to their metadata, to determine an Offset_{target}.

Additionally, in some embodiments, the estimator 1000 can incorporate any other dynamics deemed relevant to the estimator updates of motor offset. Such dynamics can include but are not limited to: motor inertia and damping, friction, cable dynamics, temperature, and behavior of other cables, motors, and/or joints. Further, these dynamics can be used to help linearize the cable force-length model by improving the estimates of the cable tension and/or cable stretch, so that these dynamics need not be incorporated directly into the force-length estimation described above. Any cable force-length model can be used for these purposes, as long as it can estimate cable force from the motor angle and other sensor data.

FIGS. 12A-12D summarize several steps that can be used to extract the relatively linear force-length relationship expected from a cable in the presence of significant hysteresis and noise. Specifically, in FIGS. 12A-12D, the x-axes represent length of cable stretch (millimeter) and the y-axes represent motor load (Newton).

FIG. 12A is a diagram illustrating a desired linear relationship 1200 to be extracted from data indicated by the multiple plotted dots 1210.

FIG. 12B is a diagram illustrating a bounding box 1220 for filtering out extremes in cable force and in cable stretch, where the data inside the box 1220 is used for estimation.

FIG. 12C is a diagram illustrating a result of filtering with several filters. For example, 15 Hz low-pass filter can be used on cable force, motor load can be >0, and identifying loads that are increasing (F(n)-F(n−1)>0 can be taken. A hysteresis filter can be used to detect the top of the hysteresis band indicated by lines 1230. Data when not at the top of this hysteresis band can be ignored.

FIG. 12D is a diagram illustrating the same filtering as FIG. 12C but with a further constraint on a force-length slope. For example, the force-length slope can be computed between consecutive data points to be within a desired range (e.g., between about 20 and about 55 N/mm). Note that over the course of the data collection, the cable's rest-length increased, causing the cable stretch to increase over time for a given motor load (torque) (used here as a substitute for cable force). Thus, the value of offset in this example can be expected to increase over time as new data comes in. As illustrated in FIG. 12D, a filtered portion 1240 obtained by filtering steps as describe herein can be considered.

In FIG. 12D, the filtering can be as follows:

In a first step, a low-pass filter can be used to filter cable force data to remove noise;

In a second step, only data, where the cable force is increasing for the given sample (F(n)-F(n−1)>0), can be considered, where F is the cable force;

In a third step, samples, where force <0, can be ignored;

In a fourth step, samples, where the local slope (instantaneous or averaged over some time) is outside a desired range (e.g., 20-55 N/mm), can be ignored;

In a fifth step, samples, where a hysteresis filter (illustrated in FIG. 13) indicates that the present motor angle is not at the top of the hysteresis band, can be ignored; and

In a sixth step, it can be desired to use a moving average of the incoming sensor data to focus on more recent data. This can be done by explicitly filtering the incoming sensor data or estimates, such as with a moving average. It can also be done by using algorithms that simultaneously provide real-time estimates of multiple values, such as recursive least squares.

In particular, the above steps are described for illustration purposes and the filtering is not limited by these example steps. Each step is a preferred method to improve the filtering and the filtering may or may not be performed in an exact order.

A hysteresis filter can represent hysteresis in a relationship between motor angle and joint angle with a deadzone and can indicate when the motor angle is at the top of the deadzone. The deadzone represents a region in which any motion of the motor angle does not affect the joint angle, potentially as a result of a combination of cable compliance and static friction, or otherwise backlash between the motor angle and joint angle. In other words, the hysteresis filter returns whether the motor is in a position to make the robotic joint start to move (top of the deadzone), or whether it is simply stretching the cable and developing force insufficient to overcome joint friction (not at the top of the deadzone).

FIG. 13 is a diagram illustrating an example hysteresis filter 1300. In some embodiments, inputs to the control loops may be filtered by the hysteresis filter 1300. The hysteresis filter 1300 can maintain a deadzone model that keeps track of an output Y, and updates Y when an input X is at either end of a deadzone 1310 having a positive end 1312 and a negative end 1314. The positive end 1312 of the deadzone 1310 is the side toward positive X, positive Y. The deadzone 1312 can be a total space where a vertical bar 1320 can travel. A X bar 1320 can hit one side or the other side of the deadzone 1310. The X bar 1320 can start moving the Y structure in the same direction, but otherwise the Y structure does not move (if the X bar 1320 is moving within the deadzone 1310 without touching either end).

If the X bar is at the positive side 1312 of the deadzone 1310, the Y structure is updated to increase its position along with the X bar; if the X bar is at the negative side 1314 of the deadzone 1310, the position of the Y structure is accordingly decreased. The hysteresis filter 1300 can return whether the X bar is in a position to push the Y structure in the positive direction (this happens when the X bar is at the positive end 1312 of the deadzone 1310)

### Parallel Control Scheme

A parallel control scheme as taught herein can be employed with any of the surgical robotic systems taught above or any other suitable surgical robotic system. Further, some embodiments taught herein can be employed with semi-robotic endoscopic surgical systems that are only robotic in part. In this control scheme, a motor is an antagonist when its torque command from a controller is being rectified to zero, otherwise it is an agonist.

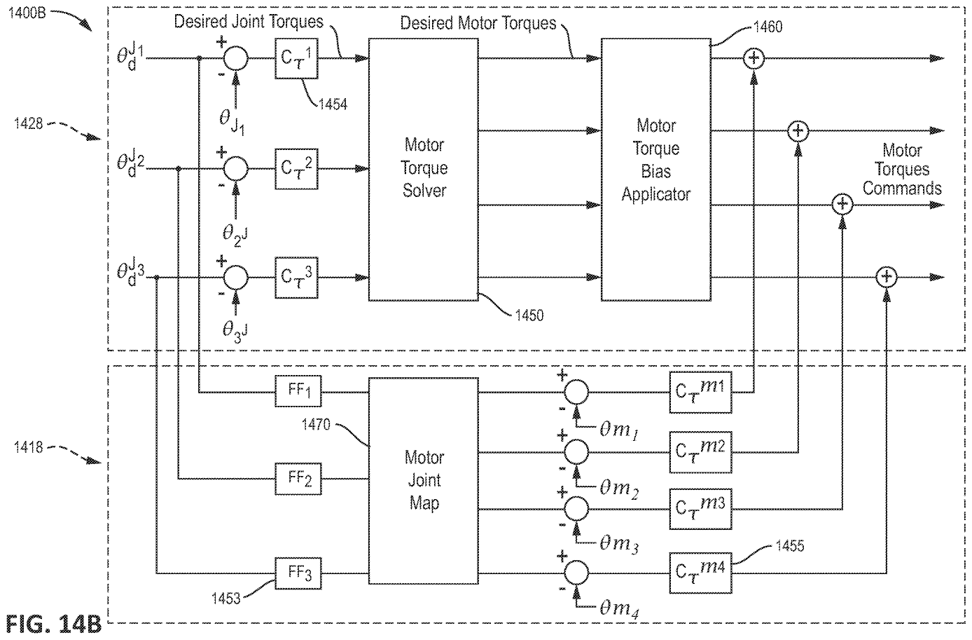

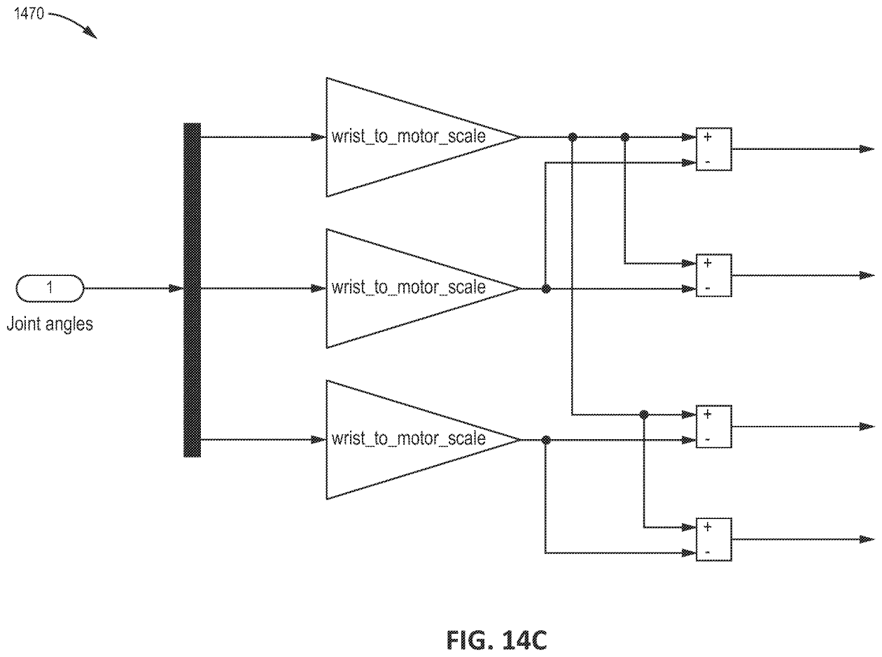

The parallel control scheme as taught herein can be understood with reference to embodiments depicted in FIG. 14A as described below. For convenience, like reference numbers are used to reference similar features of the various embodiments illustrated in the figures, unless otherwise noted.

FIG. 14A is a diagram illustrating a parallel control scheme 1400A of a joint 740 through a feedforward control loop 1410 (in a dashed box) in parallel to a motor torque control loop 1420 (in a dotted box) for each motor 710/720 in accordance with some embodiments.

The feedforward control loop 1410 can apply additional torque to each motor 710/720 to drive it toward a motor position that can achieve a desired joint angle. This parallel path can tune a controller 1430 that takes in present motor position 1434, along with a feedforward joint command that is mapped to motor space by the feedforward block 1412 to become the feedforward motor command 1436 and outputs a torque command 1432 to each motor 710/720 to drive each motor 710/720 to a position commanded by the feedforward control loop 1410. Controller 1424 calculates a torque 1422 to apply to joint 740 based on the joint error to counter external disturbances. This torque can only be generated through tension on one of the cables driven by motor #1 710 or motor #2 720 depending on the direction of the disturbance, so opposing saturation blocks 1426 isolate the torque command to a single motor. If desired, an additional co-contraction can be commanded with Fbias 1050. The feedforward torque command 1432 can be combined with a feedback torque command 1422 from closed-loop joint controller 1424. By tuning controllers 1430 and 1424 for both loops 1410 and 1420, the parallel control scheme 1400A can allow both torque mode control 1420 and feedforward control 1410 of the robotic joint. In addition, the parallel control scheme 1400A can directly control an amount of force they apply on a cable they drive and allow a user to specify how much cable tension is applied during use.

In some embodiments, one or more filters may be applied to the reference signals that go to the respective control loops to separate the frequency content to which each control loop is most sensitive. In some embodiments, a high-pass filter, or mathematically equivalent adaptive filter, is applied to the parallel position control loop error signal 1438. In some embodiments, a low-pass filter is also applied to the error signal for the torque control loop via the controller 1424. The cutoff frequency of these filters should be set above the expected frequencies that the cables are expected to creep and/or otherwise inelastically deform. This way, the parallel position-loop path responds mainly to fast command changes, whereas the torque loop is allowed to respond and adjust accordingly to cable creep.

In some embodiments, a motor dynamical model can be used to better estimate and/or enforce an actual cable tension, as opposed to enforcing electromagnetic torque at the motor rotor. In addition, a model of cable dynamics can also be used to account for friction and other effects that affect how torque at the motor translates to cable force at the robotic joint.

FIG. 14B is a diagram illustrating another parallel control scheme 1400B with multiple coupled joints using a motor torque solver 1450, a motor torque bias applicator 1460, and a mapping function 1470. For the position mode path of the parallel scheme, the mapping function 1470 determines the motor positions from the desired joint positions with the assumption that the cables are of fixed-length. For the torque mode path of the parallel scheme 1428, the motor torques must be calculated from the desired joint torques (or forces). Often in cable-driven systems one or more joints or outputs are over-actuated (have more motor inputs than joint degrees of freedom). These systems have Jacobians that are non-square, and therefore non-invertible. The motor torque solver 1450 and motor torque bias applicator 1460 represent at least one solution for solving for the motor torques, as taught herein. The motor torque solver 1450 solves for a set of possible motor torques, based on a modified Jacobian that is invertible. The motor torque bias applicator 1460 includes an additional constraint(s) to fully specify the motor torques to achieve the desired joint torques (or forces). In the motor torque solver 1450, a modified Jacobian is created by imposing linear relationship(s) on two or more inputs (motor torques). A desired choice of the imposed relationship(s) eliminates appropriate columns from the Jacobian, making it a square, invertible matrix. The desired joint torques and the modified Jacobian are used to solve for a relationship among a subset of the motor torques. Then, the additional motor torque bias applicator 1460 produces a unique solution for all motor torques by applying additional constraint(s) that narrows the chosen input relationship(s) to a single condition. As taught herein, the motor torque bias applicator 1460 solves for all motor torques uniquely by imposing the constraint that the minimum cable tension of all cables always be equal to a desired co-contraction value.

In particular, FIG. 14C shows an example of the mapping function 1470, wherein the mapping function 1470 assumes that all radii are equal and has a value of wrist_to_motor_scale. The outputs of the mapping function 1470 are cable tensions.

The scheme 1400B also includes FFn blocks 1453, which are feedforward terms as applied to each of the joints, as well as closed-loop joint controller 1454 similar to the closed-loop joint controller 1424 in FIG. 14A and tuning controllers 1455 similar to the tuning controllers 1430 in FIG. 14A.

### Computing Module

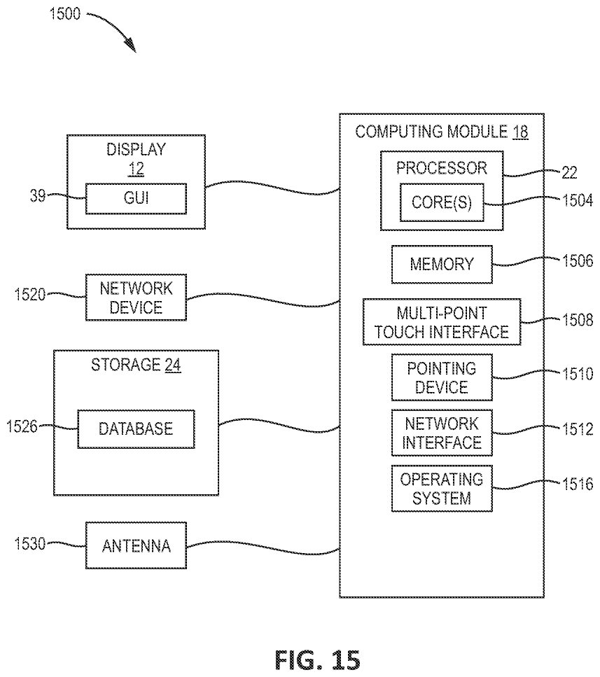

FIG. 15 schematically depicts the computing module 18 in more detail. Computing module 18 can be used to perform any of the control modes or schemes as described herein as well as one or more steps of the methods provided by example embodiments. The computing module 18 includes one or more non-transitory computer-readable media for storing one or more computer-executable instructions or software for implementing example embodiments. The non-transitory computer-readable media can include, but are not limited to, one or more types of hardware memory, non-transitory tangible media (for example, one or more magnetic storage disks, one or more optical disks, one or more USB flashdrives), and the like. For example, memory 1506 included in the computing module 18 can store computer-readable and computer-executable instructions or software for implementing example embodiments, for example, the models taught herein, the control modes and schemes as taught herein as well as other controls taught herein. The computing module 18 also includes the processor 22 and associated core 1504, for executing computer-readable and computer-executable instructions or software stored in the memory 1506 and other programs for controlling system hardware. The processor 22 can be a single core processor or multiple core (1504) processor.

Memory 1506 can include a computer system memory or random access memory, such as DRAM, SRAM, EDO RAM, and the like. The memory 1506 can include other types of memory as well, or combinations thereof. A user can interact with the computing module 18 through the display 12, such as a touch screen display or computer monitor, which can display the graphical user interface (GUI) 39. The display 12 can also display other aspects, transducers and/or information or data associated with example embodiments. The computing module 18 can include other I/O devices for receiving input from a user, for example, a keyboard or any suitable multi-point touch interface 1508, a pointing device 1510 (e.g., a pen, stylus, mouse, or trackpad). The keyboard 1508 and the pointing device 1510 can be coupled to the visual display device 12. The computing module 18 can include other suitable conventional I/O peripherals.

The computing module 18 can also include one or more storage devices 24, such as a hard-drive, CD-ROM, or other computer readable media, for storing data and computer-readable instructions, applications, and/or software that implements example operations/steps of the surgical robotic system 10 as taught herein, or portions thereof, which can be executed to generate the graphical user interface 39 on the display 12. Example storage devices 24 can also store one or more databases for storing any suitable information required to implement example embodiments. The databases can be updated by a user or automatically at any suitable time to add, delete or update one or more items in the databases. Example storage device 24 can store one or more databases 1526 for storing provisioned data, and other data/information used to implement example embodiments of the systems and methods taught herein.

The computing module 18 can include a network interface 1512 configured to interface via one or more network devices 1520 with one or more networks, for example, Local Area Network (LAN), Wide Area Network (WAN) or the Internet through a variety of connections including, but not limited to, standard telephone lines, LAN or WAN links (for example, 802.11, T1, T3, 56 kb, X.25), broadband connections (for example, ISDN, Frame Relay, ATM), wireless connections, controller area network (CAN), or some combination of any or all of the above. The network interface 1512 can include a built-in network adapter, network interface card, PCMCIA network card, card bus network adapter, wireless network adapter, USB network adapter, modem or any other device suitable for interfacing the computing module 18 to any type of network capable of communication and performing the operations taught herein. Moreover, the computing module 18 can be any computer system, such as a workstation, desktop computer, server, laptop, handheld computer, tablet computer (e.g., the iPad® tablet computer), mobile computing or communication device (e.g., the iPhone® communication device), or other form of computing or telecommunications device that is capable of communication and that has sufficient processor power and memory capacity to perform the operations taught herein.

The computing module 18 can run any operating system 1516, such as any of the versions of the Microsoft® Windows® operating systems, the different releases of the Unix and Linux operating systems, any version of the MacOS® for Macintosh computers, any embedded operating system, any real-time operating system, any open source operating system, any proprietary operating system, any operating systems for mobile computing devices, or any other operating system capable of running on the computing device and performing the operations taught herein. In some embodiments, the operating system 1516 can be run in native mode or emulated mode. In some embodiments, the operating system 1516 can be run on one or more cloud machine instances.

The computing module 18 can also include an antenna 1530, where the antenna 1530 can transmit wireless transmissions a radio frequency (RF) front end and receive wireless transmissions from the RF front end.

### Hybrid Control Scheme

A hybrid control scheme as taught herein can be employed with any of the surgical robotic systems taught above or any other suitable surgical robotic system. Further, some embodiments taught herein can be employed with semi-robotic endoscopic surgical systems that are only robotic in part.

In the hybrid control scheme a motor's role defines whether it is being controlled in position mode (agonist) or in torque mode (antagonist). Unlike the other control schemes, in the hybrid control scheme, a motor is an antagonist only if it meets the criteria for switching to an antagonist for all joints that the motor controls. The role of a motor for a given joint may be determined in one of several ways.

In some embodiments, the hybrid control scheme as taught herein can switch motor roles based on a desired motor angle. For example, the hybrid control scheme can switch a motor from an agonist role to an antagonist role when a desired motor angle becomes spooled out past a nominal motor angle (optionally plus an offset). The hybrid control scheme can switch a motor from the antagonist role to the agonist role for the given joint when the desired motor angle becomes spooled in past a nominal motor angle (optionally plus an offset) or when the desired motor angle becomes spooled in past the present angle of the antagonist, to help keep the transition smooth.

In some embodiments, the hybrid control scheme as taught herein can switch motor roles based on a velocity of a desired motor angle. For example, the hybrid control scheme can switch motor roles when the sign of the velocity of the desired motor angle changes. If the velocity of the desired motor angle becomes negative, the motor is desired to spool out, and therefore its role for that joint should be switched to antagonist. Conversely, if the velocity of the desired motor angle becomes positive, the role of the motor should be switched to agonist for the given joint.

In some embodiments, the hybrid control scheme as taught herein can switch motor roles based on agonist's applied force. For example, the hybrid control scheme can monitor the agonist's force and switch to an antagonist role when the force drops to a set transition force (this transition force can be the desired co-contraction force).

In some embodiments, executing the hybrid control scheme may include executing a hybrid joint control algorithm that puts some motors in a torque mode and other in the position mode or the impedance control mode, and switches between the two modes based on a plurality of criteria. In some embodiments, executing the hybrid joint control algorithm may include automatically switching between the position mode and the torque mode based at least in part on one of any derivative of: any of a motor's desired position or torque, a motor's measured position or torque, any joint's desired position or torque, or any joint's measured position or torque

In some embodiments, during a switch, a new agonist can move to follow a desired motor angle. A new antagonist can change its applied torque from the presently applied torque to a desired co-contraction torque. Note that these motors can be in either torque or position mode during the switch, depending on the exact method of making the switch (e.g., transitions as described below).

### A. Estimation Of Previous States

The hybrid control scheme can continuously record both the agonist and antagonist positions and forces applied. Various smoothing and filtering methods can be used to provide more accurate estimates of the positions and forces of the motors. The data can be then used to help determine how the motors can behave in torque mode and/or position mode when switching. Complex estimation techniques can also be used to estimate and/or predict when motor forces will reach a target value (e.g., adaptive position-limiting as described herein). The estimated positions (relative to the nominal motor angles) and torques of the motors just prior to the transitions can be referred to as “prior-agonist position”, and “prior-antagonist torque”, respectively. These values can be held by the hybrid control scheme to determine what the motors should do when they switch roles back to the prior roles.

### B. Role Switching Transition

To avoid sudden motions or tension changes, the motor roles can be switched gradually over a transition zone or period, maintaining continuity in position and torque at each motor. The types of motor commands can switch instantly (force to position and vice versa) but the transition can help to prevent discontinuities between a previous torque and the torque for a new position, or vice versa. During a transition, a motor can gradually change from one role to another by smoothly changing their position or torque.

The transition itself can be defined by the switching methods as described above, along with a range of position, velocity, torque, or the like, over which the transition occurs. For example, if switching starts when a desired motor angle crosses a nominal motor angle, the transition can last until the desired motor angle is some threshold beyond the nominal motor angle.

In some embodiments, the hybrid control scheme can make a gradual transition for motor role switching from an antagonist role to an agonist role. For example, the hybrid control scheme can record the prior-antagonist position. The hybrid control scheme can switch to position mode and initially command the motor to its present position. The hybrid control scheme can move from the present motor angle toward the desired motor angle over the transition. If the transition were to reverse before it is complete (e.g., the trigger that's causing the switching reverses), the position command can be smoothly commanded back to the prior-antagonist position, and once the prior-antagonist position is reached, the motor can be put back into the torque mode, at which point the transition will have been undone. Alternatively, the reversal can be done in a torque mode by slowly changing the motor torque from the present torque back to the co-contraction torque.

In some embodiments, the hybrid control scheme can make a gradual transition for motor role switching from an agonist role to an antagonist role. For example, the hybrid control scheme can record prior-agonist torque and prior-agonist position. The hybrid control scheme can switch to torque mode and start by commanding the prior-agonist torque. The hybrid control scheme can gradually change the torque to the desired co-contraction torque over the transition. If the transition were to reverse before it is complete, the motor can be put back into a position mode, initially commanded the present position, and smoothly commanded to move back toward the prior-agonist position, at which point the transition will have been undone.

### C. Adaptation To Cable Creep

When comparing a desired motor angle to a nominal motor angle, it can be assumed that a motor mapping does not change over time, (for example, the cable has the same overall length as it did in an initial tensioning) and therefore, a cable stretch and a cable force can be estimated from these two angles. In some embodiments, the hybrid control scheme can account for cable lengthening over time (creep or the like) based at least in part on mapping from joint to motor space that is updated over time. The cable slack may increase as the cable creeps.

Rather than assuming a fixed mapping from joint to motor space, a motor mapping can be updated over time (e.g., adaptive position-limiting as described above as a similar method of doing this update). If an antagonist appropriately maintains tension, it will be approximately in the same position relative to its nominal motor angle, at least on average, for a given amount of cable creep. As the cable creeps or otherwise inelastically stretches over time, a motor that is behaving as the antagonist can start to follow further and further behind the nominal motor angle based on the original mapping in order to achieve some desired antagonist force as it takes up the slack generated by the stretching cable. Given the use of torque mode control, the antagonist motor's cable remains under the appropriate tension, but the switching point between agonist and antagonist that is based on the computed nominal motor angle does not update as the cable creeps. Therefore, without adaptation, the hybrid control's role switching may not work as intended.

To adapt to this cable creep, the hybrid control scheme can keep track of the offset between the antagonist position and the nominal motor angle. The hybrid control scheme can gradually update the motor mapping so that the nominal motor angle approaches the antagonist position. Since this is an adaptation, it can be done slowly with respect to motor motions. This adaptation can update the nominal motor angle so that it effectively follows the antagonist position.

Note that the motor mapping (nominal motor angle) can be updated as described above, the trigger(s) for initiating/ending the switching of agonist/antagonist can also be updated, since the trigger can be based on the motor mapping.

In some embodiments, a motor dynamical model can be used to better estimate and/or enforce the actual cable tension for motor feedforward of the hybrid control scheme, as opposed to just enforcing electromagnetic torque at the motor rotor. In addition, a model of cable dynamics can also be used to account for friction and other effects that affect how torque at a motor translates to a cable force at a robotic joint.

In some embodiments, the hybrid control scheme can allow for a backlash compensation method. Rather than commanding a new agonist motor to a position where it produces the co-contraction force, the new agonist can be moved to a position where it generates almost enough cable force to start moving a robotic arm. The amount of position-change to achieve the above force requirement can either be pre-set (e.g. just by adding the position offset generated from an independent backlash compensator), or can be learned over time by the hybrid control scheme that keeps track of motor forces and positions as motors are driven back and forth. A pre-tuned value can be used as an initial guess, and then the hybrid control scheme can adjust based on further observations. Limits on this adjustment can be included to prevent the hybrid control scheme from making too big of a change if it is in error. One possible implementation of this is that a backlash compensation term may simply be applied to a desired motor angle.

In some embodiments, the impedance control mode may be used as part of any of the above-referenced control schemes for cable robot control of the position-limiting scheme, the parallel control scheme, or the hybrid control scheme. In particular, the impedance control mode may be an addition to or replacement for the torque mode or the position mode in any one of the position-limiting scheme, the parallel control scheme, and the hybrid control scheme.

In impedance control, there is a control law that provides a relationship between any combination of (setpoint, sensed position, any other reference position or positions) and the force or torque applied. The reference position(s), if used, could be a fixed value, or could be changing as a function of any other number of inputs. For example, an impedance control mode may be used where the torque, T, applied to a rotary joint, is:

$$T = K 1 * ( theta\_setpoint - theta\_sensed ) + B 1 * ( theta\_sensed \_dot ) + K 2 * ( theta\_sensed - theta\_reference ) + B 2 * ( theta\_setpoint \_dot - theta\_sensed \_dot )$$

### Where:

theta_setpoint is the setpoint angle
theta_sensed is the sensed angle
theta_reference is the reference angle
dot indicates a time derivative

The position control scheme may be a special case of impedance control (as described herein). That is, all terms are a derivative of the error between the setpoint and the sensed position and theta_reference is not used. If, for example, a PID controller was used to control a motor in position mode with the I term set to 0, this would be the same as the above example where B1 and K2 are set to 0 (making K the proportional gain and B2 the derivative gain)

Impedance control can be used as part of any of the three claimed options for cable robot control, including the position-limiting scheme, the parallel control scheme, and the hybrid control scheme. For example, in the position-limiting scheme, impedance control can be used to limit the motor angle to the switching angle in a way that is not absolute (i.e. the motor could be allowed to interact with the switching angle in a manner consistent with an impedance(s) selected for the impedance control law: the switching angle can be made to be the reference angle). In the parallel scheme, there can be an additional impedance control path that produces desired motor torques as a function of desired joint angles, and these torques can be added to the motor torques from the other two paths illustrated in FIG. 14A. In the hybrid scheme, impedance control can be used instead of either the position control or the torque control. In general, impedance control can be used instead of position control in many cases, depending on the desired behavior.

1. A surgical robotic system comprising:
a robotic joint of a robotic arm;
a first motor;
a second motor;
a cable coupled to the first motor and the second motor and operationally coupled to the robotic joint to control a position of the robotic joint;
a memory storing one or more instructions;
a processor configured to or programmed to read the one or more instructions stored in the memory, the processor configured to:
execute the one or more instructions in the memory to adjust the first motor and/or the second motor to remove an unacceptable amount of slack in the cable.

2. The system of claim 1, wherein executing the one or more instructions in the memory to adjust the first motor and the second motor to remove the unacceptable amount of slack in the cable comprises:
executing a position-limiting scheme;
executing a parallel control scheme; or
executing a hybrid control scheme.

3. The system of claim 2, wherein executing the one or more instructions in the memory to adjust the first motor and the second motor to remove the unacceptable amount of slack in the cable further comprises adjusting a motor angle associated with the first motor or the second motor based at least in part on the motor offset.

4. The system of claim 2, wherein executing the one or more instructions in the memory to adjust the first motor and the second motor to remove the unacceptable amount of slack in the cable further comprises adaptively to update the model in real time based at least in part on the motor offset.

5. The system of claim 2, wherein executing the one or more instructions in the memory to adjust the first motor and the second motor to remove the unacceptable amount of slack in the cable further comprises updating the model over time based at least in part on incoming cable tension and/or cable stretch.

6. The system of claim 2, wherein the hybrid control scheme accounts for cable lengthening over time based at least in part on a mapping from joint to motor space that is updated over time.

7. The system of claim 2, wherein the processor is further configured to:
receive a control scheme selection input; and
execute a control scheme.

8. The system of claim 7, wherein the control scheme is a position-limiting scheme, wherein executing the control scheme comprises:
determining a desired joint angle associated with the first motor and the second motor; and
adjusting the angle of the first motor and the second motor to drive the robotic joint to reach the respective desired joint angle.

9. The system of claim 7, wherein the control scheme is a parallel control scheme, wherein executing the parallel control scheme comprises executing a joint control algorithm based on a motor torque control loop with a parallel path of a feedforward control loop for position control or impedance control of motors to enable cable tension maintenance from the motor torque loop as well as feedforward response time from the feedforward control loop, wherein executing the joint control algorithm comprises:
determining a respective desired motor torque associated with the first motor and the second motor based at least in part on a torque control loop and a feedforward control loop in parallel to the torque control loop, the feedforward control loop providing additional motor torques to the first motor and the second motor to drive the first motor and the second motor toward a respective desired motor angle associated with the first motor and the second motor; and
adjusting a respective motor torque associated with the first motor and the second motor to the respective desired motor torque.

10. The system of claim 7, wherein the control scheme is a hybrid control scheme, wherein executing the control scheme comprises executing a hybrid joint control algorithm that puts some motors in a torque mode and others in a position mode or an impedance control mode, and switches between the two modes based on some criteria, wherein executing the hybrid joint control algorithm comprises:
automatically switching between the position mode and the torque mode based at least in part on one of any derivative of: any of a motor's desired position or torque, a motor's measured position or torque, any joint's desired position or torque, or any joint's measured position or torque.

11. The system of claim 9, wherein inputs to the feedforward and torque control loops are filtered.

12. The system of claim 1, wherein executing the one or more instructions in the memory to adjust the first motor and the second motor to remove the unacceptable amount of slack in the cable further comprises executing a robotic joint control algorithm that includes a slack avoidance algorithm that allows for a controlled amount of slack (positive or negative) for motors in position control or impedance control.

13. The system of claim 12, wherein executing the one or more instructions in the memory to adjust the first motor and the second motor to remove the unacceptable amount of slack in the cable further comprises performing an adaptive slack avoidance that adjusts the slack avoidance algorithm's parameters based on sensor data and associated metadata.

14. The system of claim 1, further comprising one or more additional motors, wherein the processor is further configured to execute the one or more instructions in the memory to adjust the one or more additional motors to remove the unacceptable amount of slack in one or more cables.

15-30. (canceled)
